SEQUENCE LISTING (2 sequences)

SEQ ID NO: 1 - DNA, 1401 bp, Artificial Sequence
  834 molecule nucleic acid sequence
atggtcagctactgggacaccggggtcctgctgtgcgcgctgctcagctgtctgcttctcacaggatctagttcaggttcgcgaagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatcgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaagaattcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 2 - protein, 467 aa, Artificial Sequence
  834 molecule amino acid sequence
MVSYWDTGVLLCALLSCLLLTGSSSGSRSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKEFEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2015/0150796 A1
(43) Pub. Date: Jun. 4, 2015

(54) Cryopreserved Implantable Cell Culture Devices and Uses Thereof

(71) Applicant: Neurotech USA, Inc., Cumberland, RI (US)

(72) Inventors: John D. Duggan, JR., Hinsdale, NH (US); Crystal Cortellessa, Uxbridge, MA (US)

(21) Appl. No.: 14/404,177

(22) PCT Filed: May 30, 2013
(86) PCT No.: PCT/US2013/043416

§ 371 Date: Nov. 26, 2014

Related U.S. Application Data
(60) Provisional application No. 61/653,191, filed on May 30, 2012.

(51) Int. Cl. A61K 9/00; A61L 27/18; A61L 27/48; A61L 27/56; A61K 35/30; A61L 27/38

(52) U.S. Cl. CPC A61K 9/0051; A61K 35/30; A61L 27/3869; A61L 27/3813; A61L 27/48; A61L 27/56; A61L 27/18; A61L 2430/16

(57) ABSTRACT

The invention provides cryopreserved encapsulated cell therapy devices that are capable of delivering biologically active molecules as well as methods of using these devices.

### RELATED APPLICATIONS

This application claims priority to U.S. Ser. No. 61/653,191, filed May 30, 2012, which is incorporated herein by reference in its entirety.

### FIELD OF THE INVENTION

The present invention relates generally to the field of encapsulated cell therapy.

### BACKGROUND OF THE INVENTION

Advances in molecular biology over the last two decades have led to the discovery of many protein molecules with promising therapeutic potentials, including cytokines, neurotrophic factors, soluble receptors and anti-angiogenic antibodies and molecules. However, the value of these new molecules has not been fully realized for clinical use, mainly due to the lack of an effective delivery system. The blood-retinal barrier (BRB) prevents large molecules in the blood stream from entering the retina. Circumventing this barrier is one of the major challenges for long-term sustained delivery of proteins to the retina.

For protein delivery to the retina, the traditional approaches are quite limited. There are two options for delivering proteins to the retina, bolus injection of purified recombinant proteins and gene therapy. Bolus injection of Macugen, Lucentis or Eylea have been approved for the treatment of wet form of age-related macular degeneration. However, these agents have short half-lives and require repeated long-term administrations. Gene therapy, on the other hand, can achieve sustained expression of a given protein. However, the doses of therapeutic protein are difficult to control due to the fact that no reliable means is available to regulate the expression levels of the transgene. Furthermore, it is impossible to reverse the treatment once the gene is delivered.

Encapsulated cell technology or ECT is a delivery system that uses live cells to secret a therapeutic agent. This is usually achieved by genetically engineering a specific type of cell to overexpress a particular agent. The engineered cells are then encapsulated in semi-permeable polymer capsules. The capsule is then implanted into the target surgical site. The semi-permeable membrane allows the free diffusion of nutrients and therapeutic molecules yet prevents the direct contact of the host immune systems cells with the cells within the device.

### SUMMARY OF THE INVENTION

The current invention describes a cryopreservation process for ECT devices. The cryopreservation process allows cryopreserved ECT devices to be stored indefinitely, thereby extending shelf-life from the current range of weeks/months to potentially infinity. This invention represents a major advantage in the manufacturing, storage, distribution, and costs of goods of cell culture devices.

Cell lines (such as ARPE-19 cells) can be genetically engineered to produce a therapeutic amount of one or more biologically active molecule(s). For example, the one or more biologically active molecule(s) can be an anti-angiogenic antibodies and molecule, an anti-angiogenic antibody-scaffold or a soluble VEGF receptor or PDGF receptor, as described in WO2012/075184, which is incorporated herein by reference in its entirety. Other biologically active molecule(s) may include, but are not limited to, neurotrophins, interleukins, cytokines, growth factors, anti-apoptotic factors, angiogenic factors, anti-angiogenic factors, antibodies and antibody fragments, antigens, neurotransmitters, hormones, enzymes, lymphokines, anti-inflammatory factors, therapeutic proteins, gene transfer vectors, and/or any combination(s) thereof. In various embodiments, such molecules can include, but are not limited to, brain derived neurotrophic factor (BDNF), NT-4, ciliary neurotrophic factor (CNTF), Axokine, basic fibroblast growth factor (bFGF), insulin-like growth factor I (IGF I), insulin-like growth factor II (IGF II), acid fibroblast growth factor (aFGF), epidermal growth factor (EGF), transforming growth factor α (TGF α), transforming growth factor β (TGF β), nerve growth factor (NGF), platelet derived growth factor (PDGF), glia-derived neurotrophic factor (GDNF), Midkine, phorbol 12-myristate 13-acetate, tryophotin, activin, thyrotropin releasing hormone, interleukins, bone morphogenic protein, macrophage inflammatory proteins, heparin sulfate, amphiregulin, retinoic acid, tumor necrosis factor α, fibroblast growth factor receptor, epidermal growth factor receptor (EGFR), PEDF, LEDGF, NTN, Neublastin, VEGF inhibitors and/or other agents expected to have therapeutically useful effects on potential target tissues.

Such cell lines can be encapsulated in encapsulation cell therapy (ECT) devices using any method(s) known in the art.

Described herein are implantable cell culture devices containing a core that contains one or more of the cells and/or cell lines and a semi-permeable membrane surrounding the core, wherein the membrane permits the diffusion of molecule(s) there through it, and wherein such devices are cryopreserved (i.e., following manufacture of the device and prior to implantation). For example, the cell line in the core may include one or more ARPE-19 cell lines that are genetically engineered to produce a therapeutically effective amount of cytokines, neurotrophic factors, soluble receptors, anti-angiogenic antibodies and molecules, and/or biologically active molecule(s) that are introduced into the ARPE-19 cell by an iterative transfection process, wherein the iterative transfection process comprises one, two, or three transfections; or the cell line in the core may include one or more ARPE-19 cells genetically engineered to secrete a therapeutically effective amount of one or more cytokines, neurotrophic factors, soluble receptors, anti-angiogenic antibodies and molecules, and/or biologically active molecule(s) that is at least 10,000 ng/day/10⁶ cells. Any of the cryopreserved devices described herein may also contain ARPE-19 cells genetically engineered to secrete a therapeutically effective amount of one or more biologically active molecule(s).

The terms “capsule”, “device” and “implant” are used interchangeably herein to refer to any bioartificial organ or encapsulated cell therapy device containing genetically engineered cells and cell lines (e.g., ARPE-19 cells or cell lines). The core of such cryopreserved devices may also contain a cryoprotectant agent, which can be added to the cell culture media contained within the core.

Any cryopreservation methods known in the art can be employed. By way of non-limiting example, encapsulated cell therapy devices can be placed in cryogenic storage vials, frozen under controlled rate freezing (e.g., to a temperature of −80° C.), and finally stored in vapor phase liquid nitrogen (e.g., −190° C.) conditions.

Cryopreserved devices can be transported under vapor phase liquid nitrogen (e.g., −190° C.) conditions and/or under dry ice (e.g., −70° C.) conditions.

Any suitable cryopreservation technique(s) may be employed. By way of non-limiting example, the ECT devices of the invention can be stored in dry ice (e.g., at −70° C.), in a freezer (e.g., at −80° C.), and/or in vapor phase liquid nitrogen (e.g., −190° C.). For example, cryopreserved ECT devices according to the invention can be stored at about −70° C., about −71° C., about −72° C., about −73° C., about −74° C., about −75° C., about −76° C., about −77° C., about −78° C., about −79° C., about −80° C., about −81° C., about −82° C., about −83° C., about −84° C., about −85° C., about −86° C., about −87° C., about −88° C., about −89° C., about −90° C., about −91° C., about −92° C., about −93° C., about −94° C., about −95° C., about −96° C., about −97° C., about −98° C., about −99° C., about −100° C., about −101° C., about −102° C., about −103° C., about −104° C., about −105° C., about −106° C., about −107° C., about −108° C., about −109° C., about −110° C., about −111° C., about −112° C., about −113° C., about −114° C., about −115° C., about −116° C., about −117° C., about −118° C., about −119° C., about −120° C., about −121° C., about −122° C., about −123° C., about −124° C., about −125° C., about −126° C., about −127° C., about −128° C., about −129° C., about −130° C., about −131° C., about −132° C., about −133° C., about −134° C., about −135° C., about −136° C., about −137° C., about −138° C., about −139° C., about −140° C., about −141° C., about −142° C., about −143° C., about −144° C., about −145° C., about −146° C., about −147° C., about −148° C., about −149° C., about −150° C., about −151° C., about −152° C., about −153° C., about −154° C., about −155° C., about −156° C., about −157° C., about −158° C., about −159° C., about −160° C., about −161° C., about −162° C., about −163° C., about −164° C., about −165° C., about −166° C., about −167° C., about −168° C., about −169° C., about −170° C., about −171° C., about −172° C., about −173° C., about −174° C., about −175° C., about −176° C., about −177° C., about −178° C., about −179° C., about −180° C., about −181° C., about −182° C., about −183° C., about −184° C., about −185° C., about −186° C., about −187° C., about −188° C., about −189° C., and/or about −190° C. or more (or any combination(s) thereof).

Cryopreserved devices can be thawed using any method(s) known in the art prior to use.

In some, non-limiting embodiments, the one or more biologically active molecule(s) (e.g., cytokines, neurotrophic factors, soluble receptors, anti-angiogenic antibodies and molecules, and/or other biologically active molecule(s)) can be introduced into the ARPE-19 cell using an iterative transfection process, as described in WO2012/075184. Specifically, the iterative transfection can be one transfection, two transfections, three transfections, or more transfections (e.g., 1, 2, 3, 4, 5, 6, 7, 8, 9, 10, or more transfections). When the iterative transfection process is one transfection, the cell line will contain one biologically active molecule(s). When the iterative transfection process is two transfections, the cell line will contain two biologically active molecule(s). Those skilled in the art will recognize that these may be the same or different biologically active molecule(s). When the iterative transfection process is three transfections, the cell line will contain three biologically active molecule(s). Again, these may be the same or different biologically active molecule(s). Those skilled in the art will recognize that the number of transfections in the iterative transfection process will determine the number of (same or different) biologically active molecule(s) in the resulting cell line.

The iterative transfection process can be used to introduce multiple copies of the same or different biologically active molecule(s) into the ARPE-19 cells.

ARPE-19 cells can be genetically engineered to produce a therapeutically effective amount of one or more cytokines, neurotrophic factors, soluble receptors, anti-angiogenic antibodies and molecules, and/or biologically active molecule(s), wherein the therapeutically effective amount is at least 10,000 ng/day/10⁶ cells (e.g., at least 15,000, 20,000, 25,000, 30,000, 35,000, 40,000, 45,000, 50,000, 55,000, 60,000, 65, 000, 70,000, 75,000, or more ng/day/10⁶ cells). Such cell lines are capable of producing this therapeutically effective amount for at least 3 months (e.g., at least 6, 9, 12, 15, 18, 21, or 24 months) or longer. Those skilled in the art will recognize that, such cell lines can be produced using an iterative transfection process. However, other methods known in the art can also be used to obtain production of this therapeutically effective amount of the one or more cytokines, neurotrophic factors, soluble receptors, anti-angiogenic antibodies and molecules, and/or biologically active molecule(s).

When the iterative transfection process is one transfection, the cell line contained in the device produces between 10,000 and 30,000 ng/day/10⁶ cells of the one or more cytokines, neurotrophic factors, soluble receptors, anti-angiogenic antibodies and molecules, and/or biologically active molecule(s). For example the cell line may produce about 15,000 ng/day/10⁶ cells. When the iterative transfection process is two transfections, the cell line contained in the device produces between 30,000 and 50,000 ng/day/10⁶ cells of the one or more cytokines, neurotrophic factors, soluble receptors, anti-angiogenic antibodies and molecules, and/or biologically active molecule(s). For example the cell line may produce about 35,000 ng/day/10⁶cells. When the iterative transfection process is three transfections, the cell line contained in the device produces between 50,000 and 75,000 ng/day/10⁶ cells of the one or more cytokines, neurotrophic factors, soluble receptors, anti-angiogenic antibodies and molecules, and/or biologically active molecule(s). For example the cell line may produce about 70,000 ng/day/10⁶ cells.

Those skilled in the art will recognize that any suitable device configuration known in the art can be cryopreserved in accordance with the methods and devices described herein. The choice of a particular device design or configuration does not affect the benefits associated with the cryopreservation of the devices.

Also provided are methods of increasing the shelf life of encapsulated cell therapy devices by cryopreserving the devices (i.e., after manufacture and prior to use). Those skilled in the art will recognize that this can be accomplished by incorporating one or more cryopreservation agents into the core of the device. By way of non-limiting example, the core may contain 10% glycerol as a cryopreservation agent.

In some embodiments, the core contains 0.25-1.0×10⁶ cells.

The core may additionally contain a matrix disposed within the semipermeable membrane. In other embodiments, the matrix includes a plurality of monofilaments, wherein the monofilaments are twisted into a yarn or woven into a mesh or are twisted into a yarn that is in non-woven strands, and wherein the cells or tissue are distributed thereon. Those skilled in the art will recognize that the monofilaments can be made from a biocompatible material selected from acrylic, polyester, polyethylene, polypropylene polyacetonitrile, polyethylene terephthalate, nylon, polyamides, polyurethanes, polybutester, silk, cotton, chitin, carbon, and/or biocompatible metals. For example, the monofilaments are polyethylene terephthalate (PET) fibers that comprises between 40-85% of the internal volume of the device.

The cell encapsulation devices described herein can also have a tether anchor. For example, the tether anchor may be an anchor loop that is adapted for anchoring the device to an ocular structure.

Once thawed, any of the devices described herein can be implanted into (or are for implantation in) the eye or another target region of the body, such as, for example, the spleen, ear, heart, colon, liver, kidney, breast, joint, bone marrow, subcutaneous, and/or peritoneal spaces. By way of non-limiting example, the devices can be implanted into (or are for implantation in) the vitreous, the aqueous humor, the Subtenon's space, the periocular space, the posterior chamber, and/or the anterior chamber of the eye.

In some illustrative embodiments, the jackets of the devices described herein are made from a permselective, immunoisolatory membrane. For example, the jackets are made from an ultrafiltration membrane or a microfiltration membrane. Those skilled in the art will recognize that an ultrafiltration membrane typically has a pore size of 1-100 nm, whereas a microfiltration membrane typically has a pore size of 0.1-10 μm. In other embodiments, the jacket may be made from a non-porous membrane material (e.g., a hydrogel or a polyurethane). The terms “jacket” and “semi-permeable membrane” are used interchangeably herein.

In some illustrative embodiments, the semi-permeable membrane of the devices described herein is made from a permselective, immunoprotective membrane. In other embodiments, the semi-permeable membrane is made from an ultrafiltration membrane or a microfiltration membrane. Those skilled in the art will recognize that a semi-permeable membrane typically has a median pore size of about 100 nm.

In still other embodiments, the semi-permeable membrane may be made from a non-porous membrane material (e.g., a hydrogel or a polyurethane). In any of the devices described herein, the nominal molecule weight cutoff (MWCO) of the semi-permeable membrane is between 50 and 500 kD (e.g., 50, 75, 100, 125, 150, 175, 200, 225, 250, 275, 300, 325, 350, 375, 400, 425, 450, 475, or 500). The semi-permeable membrane may be between about 90-120 μm (e.g. 90, 95, 100, 105, 110, 115, or 120) thick. The length of the device can be between about 1 mm-20 mm (e.g., 1, 2, 3, 4, 5, 6, 7, 8, 9, 10, 11, 12, 13, 14, 15, 16, 17, 18, 19, or 20). In some embodiments, the device has an internal diameter of between about 0.1 mm-2.0 mm (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0).

In one embodiment, the ends of the device are sealed using methyl methacrylate.

In any of the devices described herein, the capsule can be configured as a hollow fiber or a flat sheet. However, those skilled in the art will recognize that any other device configuration(s) appropriate for maintaining biological activity and for providing access for delivery of the biologically active molecule(s) can also be employed.

Moreover, in various embodiments, at least one additional biologically active molecule can be co-delivered from these devices. For example, the at least one additional biologically active molecule can be produced or released from a non-cellular or a cellular source (i.e., the at least one additional biologically active molecule is produced by one or more genetically engineered ARPE-19 cells or cell lines in the core).

By way of non-limiting example, a device for use in accordance with the instant invention may include one, two, three, four, five, six, seven or all of the following additional characteristics:
a. the core contains between 0.5-1.0×10⁶ ARPE-19 cells;
b. the length of the device is between 1 mm-20 mm;
c. the internal diameter of the device is between 0.1-2 0 mm;
d. the ends of the device are sealed using methyl methacrylate;
e. the semi-permeable membrane has a median pore size of about 100 nm;
f. the nominal MWCO of the semi-permeable membrane is 50-500 kD;
g. the semi-permeable membrane is between 90-120 μm thick;
h. the core contains an internal scaffold, wherein the scaffold comprises polyethylene terephthalate (PET) fibers that comprise between 40-85% of the internal volume of the device; and
i. any combination(s) thereof.

Those skilled in the art will recognize that, in any of the methods and uses described herein, cryopreserved devices according to the invention are preferably thawed prior to use. Any suitable method(s) for thawing such devices known in the art can be employed.

The invention further provides uses following thawing of any of the implantable cell culture devices of the invention to deliver an appropriate therapeutic dose of any biologically active molecule(s) (e.g., cytokines, neurotrophic factors, soluble receptors, anti-angiogenic antibodies and molecules, and/or other biologically active molecule(s)) to an eye of a subject. For example, the therapeutic dose is at least 100 ng/day/eye (e.g., at least 100, 200, 300, 400, 500, 1000, 1500, 2000, 2500, 3000, 3500, 4000, or more ng/day/eye).

Also provided herein are methods for treating ophthalmic disorders by thawing the cryopreserved device, implanting any of the implantable cell culture devices of the invention into the eye of a patient, and allowing soluble receptors or anti-angiogenic antibodies and molecules to diffuse from the device and bind to VEGF and/or PDGF in the eye, thereby treating the ophthalmic disorder. In some embodiments, the invention provides cell lines (i.e., any of the cell lines described herein) for use in treating ophthalmic disorders, wherein the cell lines are incorporated in an implantable cell culture device, wherein, following thawing of the cryopreserved devices, the devices are implanted into the eye of a patient, and wherein the soluble receptors or anti-angiogenic antibodies and molecules diffuse from the device and bind to VEGF and/or PDGF in the eye, thereby treating the ophthalmic disorder.

Also provided are methods for treating ophthalmic disorders by thawing the cryopreserved device, implanting any of the implantable cell culture devices of the invention into the eye of a patient, and allowing one or more cytokines, neurotrophic factors, soluble receptors, anti-angiogenic antibodies and molecules, and/or biologically active molecule(s) to diffuse from the device in the eye, thereby treating the ophthalmic disorder. For example, the invention provides cell lines (i.e., any of the cell lines described herein) for use in treating ophthalmic disorders, wherein the cell lines are incorporated in an implantable cell culture device, wherein the devices are implanted into the eye of a patient, and wherein, following thawing of the cryopreserved devices, one or more cytokines, neurotrophic factors, soluble receptors, anti-angiogenic antibodies and molecules, and/or biologically active molecule(s) diffuses from the device in the eye, thereby treating the ophthalmic disorder.

For example, the ophthalmic disorder to be treated can be selected from retinopathy of prematurity, diabetic macular edema, diabetic retinopathy, age-related macular degeneration (e.g. wet form age-related macular degeneration or atrophic AMD (also called the dry form of AMD)), glaucoma, retinitis pigmentosa, cataract formation, retinoblastoma and retinal ischemia. In one embodiment, age-related macular degeneration is wet form age-related macular degeneration. In another embodiment, the ophthalmic disorder is diabetic retinopathy.

Surprisingly, in any of the methods and uses described herein, cryopreservation does not adversely affect device output. In fact, cryopreservation can enhance the device output by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, or more.

Those skilled in the art will recognize that any of the devices described herein can also be used to treat a variety of non-ocular disorders. For non-ocular disorders, the design of the devices will have to be modified. Modification of the device design is within the routine level of skill in the art.

The invention further provides methods for inhibiting endothelial cell proliferation or vascularization by thawing the cryopreserved device, implanting the implantable cell culture device of the invention into a patient suffering from a cell proliferation disorder, and allowing the soluble receptors or anti-angiogenic antibodies and molecules to diffuse from the device and bind to VEGF and/or PDGF, wherein the binding inhibits endothelial cell proliferation or vascularization in the patient. Likewise, also provided are methods for inhibiting endothelial cell proliferation or vascularization by thawing the cryopreserved device and implanting the implantable cell culture devices of the invention into a patient suffering from a cell proliferation disorder, and allowing one or more cytokines, neurotrophic factors, soluble receptors, anti-angiogenic antibodies and molecules, and/or biologically active molecule(s) to diffuse from the device and inhibit endothelial cell proliferation or vascularization in the patient.

For example, the cell proliferation disorder may be selected from hematologic disorders, atherosclerosis, inflammation, increased vascular permeability and/or malignancy. In such methods, the therapeutically effective amount per patient per day of cytokines, neurotrophic factors, soluble receptors and anti-angiogenic antibodies and molecules, and/or biologically active molecule(s) diffuses from the device.

Also provided are methods of delivering cytokines, neurotrophic factors, soluble receptors, anti-angiogenic antibodies and molecules, and/or biologically active molecule(s) to a recipient host by thawing the cryopreserved device and implanting any of the implantable cell culture devices described herein into a target region of the recipient host, wherein the one or more encapsulated ARPE-19 cells or cell lines secrete the cytokines, neurotrophic factors, soluble receptors, anti-angiogenic antibodies and molecules, and/or biologically active molecule(s) at the target region. In other embodiments, the invention provides methods of delivering one or more biologically active molecules to a recipient host by thawing the cryopreserved device and implanting any of the implantable cell culture devices described herein into a target region of the recipient host, wherein the one or more encapsulated ARPE-19 cells or cell lines secrete the one or more biologically active molecules at the target region.

Preferred target regions can include, but are not limited to, the central nervous system, including the brain, ventricle, spinal cord, the aqueous and vitreous humors of the eye, spleen, ear, heart, colon, liver, kidney, breast, joint, bone marrow, subcutaneous, and/or peritoneal spaces. Other target regions may include, but are not limited to, whole body for systemic delivery and/or localized target sites within or near organs in the body such as breast, colon, spleen, ovary, testicle, and/or bone marrow. In such methods, the therapeutically effective amount per patient per day of cytokines, neurotrophic factors, soluble receptors and anti-angiogenic antibodies and molecules, and/or biologically active molecule(s) diffuses into the target region.

Those skilled in the art will recognize that in any of the methods and uses described herein with regard to ocular implantation and/or disorders, between 0.1 pg and 10,000 μg per patient per day of biologically active molecule(s) (e.g., cytokines, neurotrophic factors, soluble receptors, anti-angiogenic antibodies and molecules, and/or other biologically active molecule(s)) can diffuse from the implantable cell culture devices. However, for systemic implantation into other target regions of the body, the therapeutically effective amount could be upwards of 1000 mg per patient per day. For such systemic indications, those skilled in the art will recognize that far larger ECT devices would have to be employed.

For ocular implantation, the therapeutic amount is any amount between 1 pg to 10,000 μg/day/6 mm-8.5 mm device (inclusive). In some embodiments, the therapeutic amount is at least 1000 ng/day (1.0 pcd). Moreover, the cells lines and devices of the instant invention are able to express this therapeutic amount for a period of at least three weeks. In other embodiments, the therapeutic amount is at least 100-10,000 ng/day. In one non-limiting embodiment, the amount is at least 4 μg/day. When delivering soluble receptors and anti-angiogenic antibodies and molecules, delivery of 5-10 μg/day is optimal. Achieving this dosage may require the implantation of more than one device per eye. When delivering other biologically active molecule(s), it may be possible to utilize a shorter device that delivers a lower dose of the biologically active molecule(s).

The invention also provides methods for making the implantable cell culture devices of the invention. For example, by genetically engineering at least one ARPE-19 cell to secrete one or more cytokines, neurotrophic factors, soluble receptors, anti-angiogenic antibodies and molecules, and/or biologically active molecule(s).

The invention also describes the use of one or more ARPE-19 cell lines that are genetically engineered to produce one or more cytokines, neurotrophic factors, soluble receptors, anti-angiogenic antibodies and molecules, and/or biologically active molecule(s) in the manufacture of any of the implantable cell culture devices according to the invention for treating disorders including those in the eye, for example, by implantation (following thawing the cryopreserved device) of the device into the eye of the patient or at other diseased site for localized and cytokines, neurotrophic factors, soluble receptors, anti-angiogenic antibodies and molecules, and/or biologically active molecule(s) molecule delivery.

Moreover, any of the implantable cell culture devices described herein can be used for treating ophthalmic disorders by implantation (following thawing the cryopreserved device) of the device into the eye of a patient and by allowing the soluble receptors or anti-angiogenic antibodies and molecules to diffuse from the device and bind to VEGF and/or PDGF in the eye. Similarly, any of the implantable cell culture devices described herein can be used for treating ophthalmic disorders by implantation (following thawing the cryopreserved device) of the device into the eye of a patient and by allowing the one or more cytokines, neurotrophic factors, soluble receptors, anti-angiogenic antibodies and molecules, and/or biologically active molecule(s) to diffuse from the device in the eye.

Also provided are one or more ARPE-19 cells that are genetically engineered to produce one or more soluble receptors or anti-angiogenic antibodies and molecules for treating ophthalmic disorders by implantation (following thawing the cryopreserved device) of any of the implantable cell culture devices of the invention into the eye of a patient and by allowing the one or more soluble receptors or anti-angiogenic antibodies and molecules to diffuse from the device and bind to VEGF or PDGF in the eye. Moreover, the invention also provides one or more ARPE-19 cells that are genetically engineered to produce one or more cytokines, neurotrophic factors, soluble receptors, anti-angiogenic antibodies and molecules, and/or biologically active molecule(s) for treating ophthalmic disorders by implantation (following thawing the cryopreserved device) of any of the implantable cell culture devices of the invention into the eye of a patient and by allowing the cytokines, neurotrophic factors, soluble receptors, anti-angiogenic antibodies and molecules, and/or biologically active molecule(s) to diffuse from the device in the eye.

The invention also provides for the use of one or more ARPE-19 cells that are genetically engineered to produce a polypeptide (e.g., soluble receptors or anti-angiogenic antibodies and molecules) in the manufacture of an implantable cell culture device according to the invention for inhibiting endothelial cell proliferation by implantation (following thawing the cryopreserved device) of the device into the eye of a patient suffering from a cell proliferation disorder and by allowing the soluble receptors or anti-angiogenic antibodies and molecules to diffuse from the device and bind to VEGF and/or PDGF in the eye and to thereby inhibit endothelial cell proliferation in said patient. In some embodiments, the invention also provides for the use of one or more ARPE-19 cell lines that are genetically engineered to produce one or more cytokines, neurotrophic factors, soluble receptors, anti-angiogenic antibodies and molecules, and/or biologically active molecule(s) in the manufacture of an implantable cell culture device according to the invention for inhibiting endothelial cell proliferation by implantation (following thawing the cryopreserved device) of the device into the eye of a patient suffering from a cell proliferation disorder and by allowing the cytokines, neurotrophic factors, soluble receptors, anti-angiogenic antibodies and molecules, and/or biologically active molecule(s) to diffuse from the device in the eye and to inhibit endothelial cell proliferation in said patient.

Likewise, the invention also provides implantable cell culture devices of the invention for inhibiting endothelial cell proliferation by implantation (following thawing the cryopreserved device) of the device into the eye of a patient suffering from a cell proliferation disorder and by allowing the soluble receptors or anti-angiogenic antibodies and molecules to diffuse from the device and bind to VEGF and/or PDGF in the eye and thereby inhibit endothelial cell proliferation in said patient. In other embodiments, the invention also provides implantable cell culture devices of the invention for inhibiting endothelial cell proliferation by implantation (following thawing the cryopreserved device) of the device into the eye of a patient suffering from a cell proliferation disorder and by allowing the one or more cytokines, neurotrophic factors, soluble receptors, anti-angiogenic antibodies and molecules, and/or biologically active molecule(s) to diffuse from the device in the eye and inhibit endothelial cell proliferation in said patient.

Also provided herein is the use of one or more ARPE-19 cell lines that are genetically engineered to produce a polypeptide in the manufacture of an implantable cell culture device according of the invention for delivering cytokines, neurotrophic factors, soluble receptors, anti-angiogenic antibodies and molecules, and/or biologically active molecule(s) to a recipient host by implantation (following thawing the cryopreserved device) of the device into a target region of the recipient host and wherein the encapsulated one or more ARPE-19 cells secrete the cytokines, neurotrophic factors, soluble receptors, anti-angiogenic antibodies and molecules, and/or biologically active molecule(s) at the target region. Similarly, any of the implantable cell culture devices of the invention can be used for delivering cytokines, neurotrophic factors, soluble receptors, anti-angiogenic antibodies and molecules, and/or biologically active molecule(s) to a recipient host by implantation (following thawing the cryopreserved device) of the device into a target region of the recipient host and wherein the encapsulated one or more ARPE-19 cells secrete the cytokines, neurotrophic factors, soluble receptors, anti-angiogenic antibodies and molecules, and/or biologically active molecule(s) at the target region.

Moreover, one or more ARPE-19 cells that are genetically engineered to produce any polypeptides can be used for delivering cytokines, neurotrophic factors, soluble receptors, anti-angiogenic antibodies and molecules, and/or biologically active molecule(s) to a recipient host by implantation (following thawing the cryopreserved device) of any implantable cell culture devices of the invention into a target region of the recipient host and wherein the encapsulated one or more ARPE-19 cells secrete the cytokines, neurotrophic factors, soluble receptors, anti-angiogenic antibodies and molecules, and/or biologically active molecule(s) at the target region.

Also provided are any of the implantable cell culture devices described herein for use in a method of delivering one or more cytokines, neurotrophic factors, soluble receptors, anti-angiogenic antibodies and molecules, and/or biologically active molecule(s) to the eye of a subject, comprising thawing the device, wherein the thawed device is for implantation into the eye of a patient to allow the one or more soluble receptors or anti-angiogenic antibodies and molecules to diffuse from the device and bind to VEGF, PDGF, or both VEGF and PDGF in the eye, wherein the one or more cytokines, neurotrophic factors, soluble receptors, anti-angiogenic antibodies and molecules, and/or biologically active molecule(s) is for use in a method of treating ophthalmic disorders in a method for inhibiting endothelial cell proliferation or vascularization.

Those skilled in the art will recognize that the target region is selected from the central nervous system, including the brain, ventricle, spinal cord, and the aqueous and vitreous humors of the eye. Other target regions may be situated elsewhere in the body, and ECT devices placed in proximity to those regions. Regions may include, but are not limited to, spleen, ear, heart, colon, liver, kidney, breast, joint, bone marrow, subcutaneous, and peritoneal spaces.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a schematic showing the pCpGfree-vitro Expression Vector (InvivoGen) Map.

FIG. 2 shows the stability of the cell line expressing p834.

FIG. 3 shows the histological sections of p834 ECT device after 4 weeks held in a container.

FIG. 4 shows the histology of explanted p834 ECT device after three months implantation into New Zealand white rabbit eyes.

FIG. 5 shows PCD of cell lines producing 834 protein, on a mass versus potency plot. First, second and third transfection/iteration cell lines are plotted.

FIG. 6 is a sequence alignment of p834 and Aflibercept.

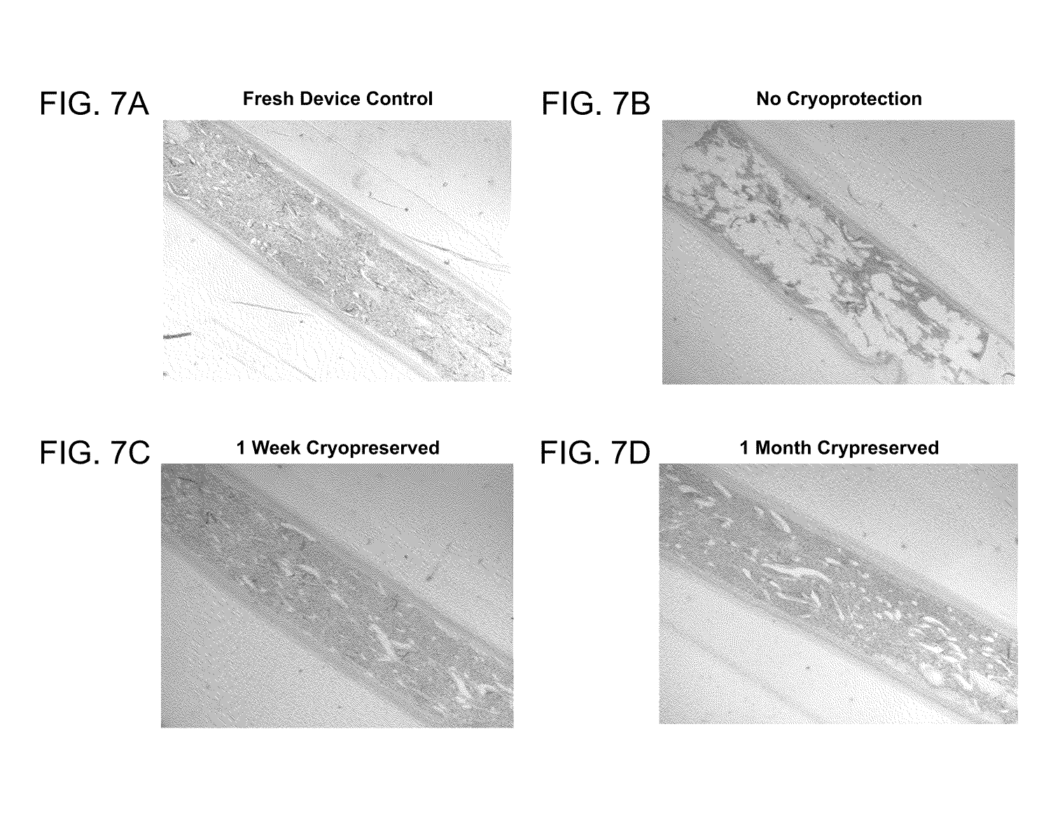

FIG. 7A is a photograph showing the histology of control cells under normal conditions one week following encapsulation without cryopreservation. FIG. 7B is a photograph showing histology of cells one week following encapsulation and frozen within vapor phase LN2 but without a cryoprotective agent formulated with the cell suspension within the device. FIG. 7C is a photograph showing histology of cells one week following encapsulation and cryopreservation in which a cryoprotective agent is formulated with the cell suspension within the device. FIG. 7D is a photograph showing the histology of cells one month following encapsulation and cryopreservation in which a cryoprotective agent is formulated with the cell suspension within the device. FIG. 7E is a photograph showing the histology of cells one year following encapsulation and cryopreservation in which a cryoprotective agent is formulated with the cell suspension within the device.

FIG. 8A is a graph showing VEGFR production from one week cryopreserved and control devices. FIG. 8B is a graph showing VEGFR production from devices, 1 month cryopreserved. FIG. 8C is a graph showing VEGFR production from devices, one year cryopreserved.

### DETAILED DESCRIPTION OF THE INVENTION

There are several advantages of ECT. First, it enables gene encoding for potentially any therapeutic protein to be engineered into the cells and therefore has a broad range of applications. The long-lasting output assures that the availability of the protein at the target site is not only continuous, but also long-term. Furthermore, the output of an ECT implant can be controlled to achieve the optimal treatment dose. Finally, the treatment by means of ECT can be terminated if necessary by simply retrieving the implant. Thus, ECT is a very effective means of long-term delivery of biologically active proteins and polypeptides to the retina. In fact, ECT has shown itself to be an excellent choice for retinal diseases, especially considering the limited therapeutic distribution volume that is required, easy access to the eye, and the chronic nature of the diseases.

However, like most cell based therapy products, ECT devices have a relative short shelf-life, in the range of several weeks to months. As a consequence, a large amount of unused product will have to be discarded. Typically, recombinant proteins, under a best case scenario, have shelf-lives in the range of 12-24 months.

Those skilled in the art will recognize that, in accordance with the present invention, any encapsulated cell therapy (ECT) devices may be cryopreserved following manufacture and prior to administration and/or implementation. Cryopreservation, if successful, helps to improve the shelf-life of the ECT devices, which, in turn, would improve device storage and/or simplify device manufacturing.

The main components of a medical therapy value chain are the ease of product manufacture, long term product expiration, and stability of product during distribution. Current methodologies for cell based therapies center on transport of living cells under conditions mimicking optimum growth conditions. These conditions may encompass, for example, control of humidity, CO₂, and temperature, and must be implemented during cell expansion, storage and distribution. These environmental parameters lead to sophisticated packaging requirements and constant environmental monitoring at each step of process post-manufacture. As shown below, cell therapy products typically have a shelf-life of a few days to a few weeks. Thus, complicated packaging requirements and short shelf-life present major challenges associated with manufacturing, storage and distribution of cell-based products.

| Company | Product | Use | Shelf Life |
|---|---|---|---|
| Organogenesis | Apligraf | Epidermal | 10 days |
| | | Cell Product | (increased from 5 days) |
| BioHeart | Myocell | Myoblast Cell | 4 days |
| | | Product | (attempting to increase |
| | | | to 7 days) |
| Histogenics | Neocart | Cartilage Cell | 5 days |
| | | Product | (attempting to increase |
| | | | to 10 days) |
| Pervasis | Vascugel | Endothelial | 14 days |
| Therapeutics | | Cell Product | (attempting to increase |
| | | | to 21 days) |
| Neurotech | NT-501 | ARPE Cell | 60 days |
| | | Product | |

The limited shelf-lives of these current cell-based products have an impact on product distribution and delivery. Thus, there is a need for cell-based products having an increased shelf-life, which will simplify manufacturing logistics.

Cryopreservation would alleviate such restraints. In this context, cryopreservation would significantly extend product shelf-life and simplify product distribution and supply to the end user, which, in turn, would result in reduced costs associated with the manufacture and distribution of ECT devices. Surprisingly, as described in detail in Example 5, infra, cryopreservation of the devices does not exert any negative or otherwise adverse effects on device output.

Any suitable cryopreservation known in the art can be used to cryopreserve any of the ECT devices described herein.

For example, cryopreservation in vapor phase liquid nitrogen is an established method for long term storage of living cells, and is dependent on appropriate cryoprotectant agents and the ability of cells to survive ultra-low temperature conditions. Once optimal conditions are met for cryopreservation, cells may be stored nearly indefinitely within vapor phase liquid nitrogen.

One advantage of ECT is that the cellular implant is self-contained within the device capsule, and no external culturing of cells is required after ECT devices are filled with cells. Thus, the ability to cryopreserve and store the entire ECT device including the cells is an attractive alternative to storage under environmentally controlled conditions.

Any suitable cryopreservation methods known in the art can be adapted to ECT products and will simplify the process of ECT device manufacture, storage and distribution. By way of non-limiting example, using a cryopreservation system, any of the ECT devices of the invention can be filled with cells formulated with cryoprotectant agent (e.g., 10% glycerol), placed in cryogenic storage vials, frozen under controlled rate freezing (e.g., to −80° C.), and finally stored in vapor phase liquid nitrogen (e.g., −190° C.) conditions. However, any other cryopreservation method(s) known in the art can also be used in accordance with the instant invention. Determination of the appropriate cryopreservation method(s) is within the routine level of skill in the art.

In addition, because the entire supply chain is simplified, any of the ECT devices of the invention can be transported under vapor phase liquid nitrogen (−190° C.) conditions and/or dry ice (−70° C.) conditions (or any combination(s) thereof).

Cryopreserved devices can be thawed using any suitable method or protocol known in the art prior to use. Surprisingly, thawed ECT devices, after one week, one month, and one year intervals under cryopreserved conditions, contain cells exhibiting robust growth and output of recombinant protein. In fact, in some instances, device output for the cryopreserved devices was improved (i.e., better or elevated) as compared to the non-cryopreserved devices at the same time points. It was not expected that the ECT device, including the materials used for capsule construction, plus the cellular contents, could withstand the ultra-low temperature conditions of vapor phase liquid nitrogen. However, the intact components of ECT devices, the healthy proliferation of cells within ECT devices after cryopreservation, and robust recombinant protein secretion from ECT devices demonstrates the durability of the ECT design used within this study. (See, Example 5, infra).

Thus, ECT cryopreservation (by any means known in the art) will enable large scale manufacture of ECT products, while significantly simplifying storage and distribution of commercially viable final products.

Proteins are a dominant class of therapeutics used in the treatment of eye diseases. However, large antibody based protein drugs are unable to bypass the blood-retinal bather and, thus, require repeated intraocular administration for treatment. It has previously been demonstrated encapsulated cell technology (ECT) intraocular devices can deliver a biotherapeutic (e.g., a biologically active molecule) directly to the eye consistently over the course of 2 years in human clinical trials, thereby suggesting this technology may be extended to other ophthalmic biologics as well, for example those related to wet AMD.

Anti-angiogenic antibody-scaffolds and anti-angiogenic molecules that can be used in the claimed invention are described in WO2012/075184, which is herein incorporated by reference. Other biologically active agents that can be used in connection with this invention include, but are not limited to, neurotrophins, interleukins, cytokines, growth factors, anti-apoptotic factors, angiogenic factors, anti-angiogenic factors, antibodies and antibody fragments, antigens, neurotransmitters, hormones, enzymes, lymphokines, anti-inflammatory factors, therapeutic proteins, gene transfer vectors, and/or any combination(s) thereof. Non-limiting examples of such molecules can include, but are not limited to, brain derived neurotrophic factor (BDNF), NT-4, ciliary neurotrophic factor (CNTF), Axokine, basic fibroblast growth factor (bFGF), insulin-like growth factor I (IGF I), insulin-like growth factor II (IGF II), acid fibroblast growth factor (aFGF), epidermal growth factor (EGF), transforming growth factor α (TGF α), transforming growth factor β (TGF β), nerve growth factor (NGF), platelet derived growth factor (PDGF), glia-derived neurotrophic factor (GDNF), Midkine, phorbol 12-myristate 13-acetate, tryophotin, activin, thyrotropin releasing hormone, interleukins, bone morphogenic protein, macrophage inflammatory proteins, heparin sulfate, amphiregulin, retinoic acid, tumor necrosis factor α, fibroblast growth factor receptor, epidermal growth factor receptor (EGFR), PEDF, LEDGF, NTN, Neublastin, VEGF inhibitors and/or other agents expected to have therapeutically useful effects on potential target tissues.

An iterative transfection process of one, two, three or more transfections (e.g., 4, 5, 6, 7, 8, 9, 10, or more) can be used to genetically engineer the cells. Surprisingly, an iterative DNA transfection and selection significantly increases the ability of cell lines to produce recombinant protein secretion from 50,000 to greater than 70,000 ng/million cells/day (70 pcd). The iterative transfection process can be used to introduce multiple copies of the same or different biologically active molecule(s) into the cells (e.g., ARPE-19 cells). Molecules produced with an iterative transfection process involving one transfection can be referred to as “first generation” molecules. Molecules produced with an iterative transfection process involving two transfections can be referred to as “second generation” molecules. Molecules produced with an iterative transfection process involving three transfections can be referred to as “third generation” molecules.

Those skilled in the art will recognize that this iterative transfection process can be used with any cytokines, neurotrophic factors, soluble receptors, anti-angiogenic antibodies and molecules, anti-angiogenic antibody-scaffolds, anti-angiogenic molecules, and/or other biologically active molecule(s).

ECT devices may be an effective drug delivery platform for large biologic molecules including antibodies, antibody scaffolds, other biologically active molecule(s) and/or receptor fusion proteins for ophthalmic indications, as well as localized and/or systemic indications.

A gene of interest (i.e., a gene that encodes a given cytokine, neurotrophic factor, soluble receptor, anti-angiogenic antibody and molecule, and/or biologically active molecule(s)) can be inserted into a cloning site of a suitable expression vector using standard techniques known in the art.

Angiogenic antibody-scaffolds and receptor fusion proteins that are derived from (and/or are biosimilar to) known anti-VEGF compounds and bioreactive fragments thereof have previously been described. (See, e.g., WO2012/075184, incorporated herein by reference). For example, the known anti-VEGF compounds include, but are not limited to, anti-VEGF receptor fragments (i.e., Aflibercept) and/or anti-VEGF antibodies (or antigen binding fragments thereof) (i.e., Bevacizumab, DrugBank DB00112; or Ranibizumab DrugBank DB01270)). The sequences of these known anti-VEGF compounds are known in the art.

One non-limiting example of a specific VEGF receptor construct that can be used in the devices and methods disclosed herein is p834 (VEGFR-Fc#1, [RS-VEGF Receptor 1, Domain 2 and VEGF Receptor 2, Domain 3 (R1D2-R2D3)]-EFEPKSC-hIgG1 Fc). However, any other suitable cytokines, neurotrophic factors, soluble receptors, anti-angiogenic antibodies and molecules, and/or biologically active molecule(s) can also be used.

The output of cell lines generated based on the 834 is summarized below.

| Derived Cell Lines | PCD | Comment |
|---|---|---|
| 834-10-5 | ~15 PCD | Became the basis for second |
| | | and third generation ECT |
| | | devices |

Transfecting p834 (also p910, p969) results in the generation of high expressing clones each time.

The specific nucleotide and amino acid sequences of the p834 construct is shown below.

For the purpose of clarity, the constructs, cell lines and anti-angiogenic antibody-scaffolds and/or anti-angiogenic molecules and/or any other biologically active molecule(s) of the instant invention are identified as follows in the instant application: “pXXX” refers to a plasmid (for example, plasmid p834), “XXX-X-XX” refers to a cell line (for example, cell line 834-10-5), and “XXX” refers to a molecule (for example, molecule 834). However, those skilled in the art will recognize that any of the scaffolds and/or constructs and/or molecules and/or cell lines based on the invention may be referred to, identified, and/or demarcated interchangeably herein.

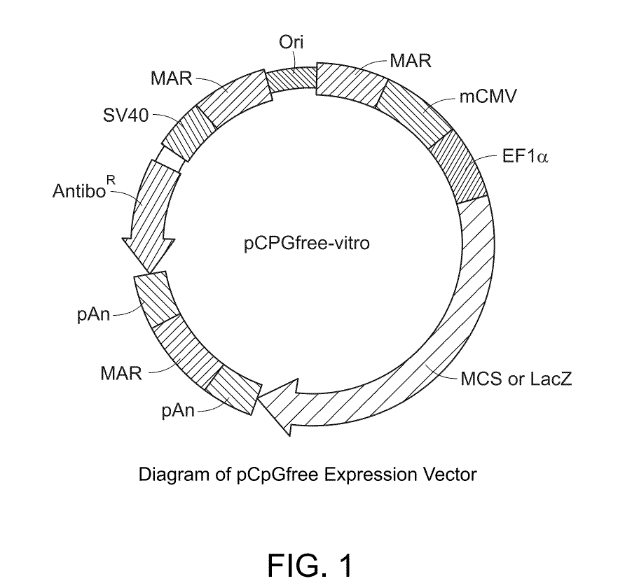

The same molecule can be introduced into different expression vectors, thereby making different plasmids. For example, molecule 834 cDNA can be introduced into pCpG vitro free blasticidin resistant vector (see FIG. 1) to make plasmid p834 cDNA. Alternatively, molecule 834 can also be introduced into pCpG vitro free neomycin resistant vector to make plasmid p910; or into pCpG hygromycin resistant vector to make plasmid p969 (See, WO2012/075184).

Using the iterative transfection process described herein, multiple copies of the same (or different) anti-angiogenic antibody-scaffolds and/or anti-angiogenic molecules and/or any other biologically active molecule(s) can be incorporated into a cell (e.g., an ARPE-19 cell). For example, when the iterative transfection process introduces two transfections, a second generation construct (910) is generated, which contains two copies of the 834 cDNA. Similarly, when the iterative transfection process introduces three transfections, a third generation construct (969) is generated, which contains three copies of the 834 cDNA.

p834

(SEQ ID NO: 1)
atggtcagctactgggacaccggggtcctgctgtgcgcgctgctcagctgtctgcttctcacaggatc
tagttcaggttcgcgaagtgatacaggtagacctttcgtagagatgtacagtgaaatccccgaaatta
tacacatgactgaaggaagggagctcgtcattccctgccgggttacgtcacctaacatcactgttact
ttaaaaaagtttccacttgacactttgatccctgatggaaaacgcataatctgggacagtagaaaggg
cttcatcatatcaaatgcaacgtacaaagaaatagggcttctgacctgtgaagcaacagtcaatgggc
atttgtataagacaaactatctcacacatcgacaaaccaatacaatcatcgatgtggttctgagtccg
tctcatggaattgaactatctgttggagaaaagcttgtcttaaattgtacagcaagaactgaactaaa
tgtggggattgacttcaactgggaatacccttcttcgaagcatcagcataagaaacttgtaaaccgag
acctaaaaacccagtctgggagtgagatgaagaaatttttgagcaccttaactatagatggtgtaacc
cggagtgaccaaggattgtacacctgtgcagcatccagtgggctgatgaccaagaagaacagcacatt
tgtcagggtccatgaaaaagaattcgagcccaaatcttgtgacaaaactcacacatgcccaccgtgcc
cagcacctgaactcctggggggaccgtcagtcttcctcttccccccaaaacccaaggacaccctcatg
atctcccggacccctgaggtcacatgcgtggtggtggacgtgagccacgaagaccctgaggtcaagtt
caactggtacgtggacggcgtggaggtgcataatgccaagacaaagccgcgggaggagcagtacaaca
gcacgtaccgtgtggtcagcgtcctcaccgtcctgcaccaggactggctgaatggcaaggagtacaag
tgcaaggtctccaacaaagccctcccagcccccatcgagaaaaccatctccaaagccaaagggcagcc
ccgagaaccacaggtgtacaccctgcccccatcccgggatgagctgaccaagaaccaggtcagcctga
cctgcctggtcaaaggcttctatcccagcgacatcgccgtggagtgggagagcaatgggcagccggag
aacaactacaagaccacgcctcccgtgctggactccgacggctccttcttcctctacagcaagctcac
cgtggacaagagcaggtggcagcaggggaacgtcttctcatgctccgtgatgcatgaggctctgcaca
accactacacgcagaagagcctctccctgtctccgggtaaa
(SEQ ID NO: 2)
mvsywdtgvllcallscllltgsssgsrsdtgrpfvemyseipeiihmtegrelvipcrvtspnitvt
lkkfpldtlipdgkriiwdsrkgfiisnatykeiglltceatvnghlyktnylthrqtntiidvvlsp
shgielsvgeklvinctartelnvgidfnweypsskhqhkklvnrdlktqsgsemkkflstltidgvt
rsdqglytcaassglmtkknstfvrvhekefepkscdkthtcppcpapellggpsvflfppkpkdtlm
isrtpevtcvvvdvshedpevkfnwyvdgvevhnaktkpreeqynstyrvvsvltvlhqdwlngkeyk
ckvsnkalpapiektiskakgqprepqvytlppsrdeltknqvsltclvkgfypsdiavewesngqpe
nnykttppvldsdgsfflyskltvdksrwqqgnvfscsvmhealhnhytqkslslspgk

A wide variety of host/expression vector combinations may be used to express the gene encoding the biologically active molecule(s) of interest. Long-term, stable in vivo expression is achieved using expression vectors (i.e., recombinant DNA molecules) in which the gene of interest is operatively linked to a promoter that is not subject to down regulation upon implantation in vivo in a mammalian host. Suitable promoters include, for example, strong constitutive mammalian promoters, such as beta-actin, eIF4A1, GAPDH, etc. Stress-inducible promoters, such as the metallothionein 1 (MT-1) or VEGF promoter may also be suitable. Additionally, hybrid promoters containing a core promoter and custom 5′ UTR or enhancer elements may be used. Other known non-retroviral promoters capable of controlling gene expression, such as CMV or the early and late promoters of SV40 or adenovirus are suitable. Enhancer elements may also be place to confer additional gene expression under stress environments, such as low O₂. One example is the erythropoietin enhancer which confers up-regulation of associated gene elements upon hypoxic induction.

The expression vectors containing the gene of interest may then be used to transfect the desired cell line. Standard transfection techniques such as liposomal, calcium phosphate co-precipitation, DEAE-dextran transfection or electroporation may be utilized. Commercially available mammalian transfection kits, such as Fugene6 (Roche Applied Sciences), may be purchased. Additionally, viral vectors may be used to transducer the desired cell line. An example of a suitable viral vector is the commercially available pLenti family of viral vectors (Invitrogen). Human mammalian cells can be used. In all cases, it is important that the cells or tissue contained in the device are not contaminated or adulterated. For antibody scaffold proteins requiring heavy and light chain components, dual constructs, each encoding a relevant antibody heavy or light chain, can be co-transfected simultaneously, thereby yielding cell lines expressing functional bivalent Fab and tetravalent full antibody molecules.

Exemplary promoters include the SV40 promoter and the CMV/EF1alpha promoter, as shown in FIG. 1.

Other useful expression vectors, for example, may consist of segments of chromosomal, non-chromosomal and synthetic DNA sequences, such as various known derivatives of SV40 and known bacterial plasmids, e.g., pUC, pBlueScript™ plasmids from E. coli including pBR322, pCR1, pMB9 and their derivatives. Expression vectors containing the geneticin (G418), hygromycin or blasticidin drug selection genes (Southern, P. J., In Vitro, 18, p. 315 (1981), Southern, P. J. and Berg, P., J. Mol. Appl. Genet., 1, p. 327 (1982)) are also useful. These vectors can employ a variety of different enhancer/promoter regions to drive the expression of both a biologic gene of interest and/or a gene conferring resistance to selection with toxin such as G418, hygromycin B, or blasticidin. A variety of different mammalian promoters can be employed to direct the expression of the genes for G418 and hygromycin B and/or the biologic gene of interest. The G418 resistance gene codes for aminoglycoside phosphotransferase (APH) which enzymatically inactivates G418 (100-1000 μg/μl) added to the culture medium. Only those cells expressing the APH gene will survive drug selection usually resulting in the expression of the second biologic gene as well. The hygromycin B phosphotransferase (HPH) gene codes for an enzyme which specifically modifies hygromycin toxin and inactivates it. Genes co-transfected with or contained on the same plasmid as the hygromycin B phosphotransferase gene will be preferentially expressed in the presence of hygromycin B at 50-200 μg/ml concentrations.

Examples of expression vectors that can be employed include, but are not limited to, the commercially available pRC/CMV (Invitrogen), pRC/RSV (Invitrogen), pCDNA1NEO (Invitrogen), pCI-Neo (Promega), pcDNA3.3 (Invitrogen) and GS vector system (Lonza Group, Switzerland). Other suitable commercially available vectors include pBlast, pMono, or pVitro. In one embodiment, the expression vector system is the pCpGfree-vitro expression vectors available with neomycin (G418), hygromycin, and blasticidin resistance genes (InvivoGen, San Diego, Calif.)) (See FIG. 1).

In one embodiment, the pNUT expression vector, which contains the cDNA of the mutant DHFR and the entire pUC18 sequence including the polylinker, can be used. See, e.g., Aebischer, P., et al., Transplantation, 58, pp. 1275-1277 (1994); Baetge et al., PNAS, 83, pp. 5454-58 (1986). The pNUT expression vector can be modified such that the DHFR coding sequence is replaced by the coding sequence for G418 or hygromycin drug resistance. The SV40 promoter within the pNUT expression vector can also be replaced with any suitable constitutively expressed mammalian promoter, such as those discussed above.

Those skilled in the art will recognize that any other suitable, commercially available expression vectors (e.g., pcDNA family (Invitrogen), pBlast, pMono, pVitro, or pCpG-vitro (Invivogen)) can also be used. Principal elements regulating expression are typically found in the expression cassette. These elements include the promoter, 5′ untranslated region (5′ UTR) and 3′ untranslated region (3′ UTR). Other elements of a suitable expression vector may be critical to plasmid integration or expression but may not be readily apparent. The skilled artisan will be able to design and construct suitable expression vectors for use in the claimed invention. The choice, design, and/or construction of a suitable vector is well within the routine level of skill in the art.

The sequences suitable biologically active molecule(s) that can be used in accordance with the instant invention have also been published and/or are known in the art. Other genes encoding the biologically active molecules useful in this invention that are not publicly available may be obtained using standard recombinant DNA methods such as PCR amplification, genomic and cDNA library screening with oligonucleotide probes.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al., MOLECULAR CLONING: A LABORATORY MANUAL, 2^{nd} ed., Cold Spring Harbor Laboratory, Cold Spring Harbor Laboratory Press, Cold Spring Harbor, N.Y. 1989), and other laboratory manuals.

The cell of choice is the ARPE-19 cell line, a spontaneously arising continuous human retinal pigmented epithelial cell line. However, those skilled in the art will recognize that other suitable cells, including but not limited to CHO cells, BHK cells, RPE (primary cells or immortalized cells), can also be used. The choice of cell depends upon the intended application. The encapsulated cells may be chosen for secretion of a biologically active molecule. Cells can also be employed which synthesize and secrete agonists, analogs, derivatives or fragments of the construct, which are active. Those skilled in the art will recognize that other suitable cell types may also be genetically engineered to secrete biologically active molecule(s).

To be a platform cell line for an encapsulated cell based delivery system, the cell line should have as many of the following characteristics as possible: (1) the cells should be hardy under stringent conditions (the encapsulated cells should be functional in the avascular tissue cavities such as in the central nervous system or the eye, especially in the intra-ocular environment); (2) the cells should be able to be genetically modified (the desired therapeutic factors needed to be engineered into the cells); (3) the cells should have a relatively long life span (the cells should produce sufficient progenies to be banked, characterized, engineered, safety tested and clinical lot manufactured); (4) the cells should be of human origin (which increases compatibility between the encapsulated cells and the host); (5) the cells should exhibit greater than 80% viability for a period of more than one month in vivo in device (which ensures long-term delivery); (6) the encapsulated cells should deliver an efficacious quantity of a useful biological product (which ensures effectiveness of the treatment); (7) the cells should have a low level of host immune reaction (which ensures the longevity of the graft); and (8) the cells should be nontumorigenic (to provide added safety to the host, in case of device leakage).

The ARPE-19 cell line (see Dunn et al., 62 Exp. Eye Res. 155-69 (1996), Dunn et al., 39 Invest. Ophthalmol. Vis. Sci. 2744-9 (1998), Finnemann et al., 94 Proc. Natl. Acad. Sci. USA 12932-7 (1997), Handa et al., 66 Exp. Eye. 411-9 (1998), Holtkamp et al., 112 Clin. Exp. Immunol. 34-43 (1998), Maidji et al., 70 J. Virol. 8402-10 (1996); U.S. Pat. No. 6,361,771) demonstrates all of the characteristics of a successful platform cell for an encapsulated cell-based delivery system. The ARPE-19 cell line is available from the American Type Culture Collection (ATCC Number CRL-2302). ARPE-19 cells are normal retinal pigmented epithelial (RPE) cells and express the retinal pigmentary epithelial cell-specific markers CRALBP and RPE-65. ARPE-19 cells form stable monolayers, which exhibit morphological and functional polarity.

Genetically engineered ARPE-19 cells express one or more biologically active molecule(s) to produce a therapeutic amount of the biologically active molecule(s). In some embodiments, the genetically engineered ARPE-19 cells are capable of producing at least 10,000 ng/day/10⁶ cells. These cells are capable of producing this amount for a period of at least 3 months.

These molecules can be introduced into the ARPE-19 cells using an iterative transfection process. The iterative transfection contains at least one transfection, two transfections, three transfections, or more transfections (e.g., 4, 5, 6, 7, 8, 9, 10, or more) transfections. The cell line of the instant invention can produce between 10,000 and 30,000 ng/day/10⁶cells, for example about or at least 15,000 ng/day/10⁶cells of the one or more biologically active molecule(s) when the iterative transfection is one transfection. Alternatively, the cell line can produce between 30,000 and 50,000 ng/day/10⁶cells, for example about or at least 35,000 ng/day/10⁶cells of the one or more biologically active molecule(s) when the iterative transfection is two transfections. In other embodiments, the cell line produces between 50,000 and 75,000 ng/day/10⁶cells, for example about or at least 70,000 ng/day/10⁶cells of the one or more biologically active molecule(s) when the iterative transfection is three transfections. In some embodiments, the same biologically active molecule(s) can be introduced into the cells using such iterative transfection. Alternatively, different biologically active molecule(s) are introduced into the cells in each transfection of the iterative transfection.

When the devices of the invention are used, between 10² and 10⁸ genetically engineered ARPE-19 cells, for example 0.5-1.0×10⁶ or 5×10² to 6×10⁵ genetically engineered ARPE-19 cells are encapsulated in each device. Dosage may be controlled by implanting a fewer or greater number of capsules, e.g., between 1 and 50 capsules per patient. The ophthalmic devices described herein are capable of delivering between about 0.1 pg and 10000 μg per eye per patient per day. In one non-limiting example, the therapeutic amount is 500-50,000 ng steady state per eye. In another example, the therapeutic amount is at least 10 μg/ml steady state per eye. Moreover, once thawed, cryopreserved devices of the instant invention are able to express this therapeutic amount for a period of at least three months.

Techniques and procedures for isolating cells or tissues which produce a selected product are known to those skilled in the art, or can be adapted from known procedures with no more than routine experimentation.

If the cells to be isolated are replicating cells or cell lines adapted to growth in vitro, it is particularly advantageous to generate a cell bank of these cells. A particular advantage of a cell bank is that it is a source of cells prepared from the same culture or batch of cells. That is, all cells originated from the same source of cells and have been exposed to the same conditions and stresses. Therefore, the vials can be treated as homogenous culture. In the transplantation context, this greatly facilitates the production of identical or replacement devices. It also allows simplified testing protocols, which insure that implanted cells are free of retroviruses and the like. It may also allow for parallel monitoring of vehicles in vivo and in vitro, thus allowing investigation of effects or factors unique to residence in vivo.

As used herein, the terms “individual” or “recipient” or “host” are used interchangeably to refer to a human or an animal subject.

As used herein, a “biologically active molecule” (“BAM”) is any substance that is capable of exerting a biologically useful effect upon the body of an individual in whom a device of the present invention is implanted. Anti-angiogenic antibody-scaffolds and anti-angiogenic antibodies and molecules are examples of BAMs. BAMs may include cytokines, neurotrophic factors, soluble receptors, and/or anti-angiogenic antibodies and molecules. Other suitable examples of BAMs can include, for example, neurotrophins, interleukins, cytokines, growth factors, anti-apoptotic factors, angiogenic factors, anti-angiogenic factors, antibodies and antibody fragments, antigens, neurotransmitters, hormones, enzymes, lymphokines, anti-inflammatory factors, therapeutic proteins, gene transfer vectors, and/or any combination(s) thereof. In various embodiments, such molecules can include, but are not limited to, brain derived neurotrophic factor (BDNF), NT-4, ciliary neurotrophic factor (CNTF), Axokine, basic fibroblast growth factor (bFGF), insulin-like growth factor I (IGF I), insulin-like growth factor II (IGF II), acid fibroblast growth factor (aFGF), epidermal growth factor (EGF), transforming growth factor α (TGF α), transforming growth factor β (TGF β), nerve growth factor (NGF), platelet derived growth factor (PDGF), glia-derived neurotrophic factor (GDNF), Midkine, phorbol 12-myristate 13-acetate, tryophotin, activin, thyrotropin releasing hormone, interleukins, bone morphogenic protein, macrophage inflammatory proteins, heparin sulfate, amphiregulin, retinoic acid, tumor necrosis factor α, fibroblast growth factor receptor, epidermal growth factor receptor (EGFR), PEDF, LEDGF, NTN, Neublastin, VEGF inhibitors and/or other agents expected to have therapeutically useful effects on potential target tissues.

The terms “capsule” and “device” and “vehicle” are used interchangeably herein to refer to the ECT devices of the invention.

Unless otherwise specified, the term “cells” means cells in any form, including but not limited to cells retained in tissue, cell clusters, and individually isolated cells.

As used herein a “biocompatible capsule” or “biocompatible device” or “biocompatible vehicle” means that the capsule or device or vehicle, upon implantation in an individual, does not elicit a detrimental host response sufficient to result in the rejection of the capsule or to render it inoperable, for example through degradation.

As used herein an “immunoisolatory capsule” or “immunoprotective capsule” or “immunoisolatory device” or “immunoprotective device” or “immunoisolatory vehicle” or “immunoprotective vehicle” means that the capsule upon implantation into an individual, favorably partitions the device cellular contents and minimizes the deleterious effects of the host's immune system on the cells within its core.

As used herein “long-term, stable expression of a biologically active molecule” means the continued production of a biologically active molecule at a level sufficient to maintain its useful biological activity for periods greater than one month, for example greater than three months or greater than six months. Implants of the devices and the contents thereof are able to retain functionality for greater than three months in vivo and in many cases for longer than a year, and in some cases longer than two years or more.

The terms “jacket” and “semi-permeable membrane” are used interchangeably herein.

The term “internal scaffold” is one example of a “matrix” that can be used in the devices described herein.

The “semi-permeable” nature of the jacket membrane surrounding the core permits molecules produced by the cells (e.g., metabolites, nutrients and/or therapeutic substances) to diffuse from the device into the surrounding host eye tissue, but is sufficiently impermeable to protect the cells in the core from detrimental immunological attack by the host.

The terms “encapsulated cell therapy” or “ECT” are used interchangeably herein to refer to any device capable of isolating cells from the recipient host's immune system by surrounding the cells with a semipermeable biocompatible material before implantation within the host. Those skilled in the art will recognize that in any of the devices, methods, and/or uses of the presented invention, any ECT devices known in the art can be employed.

The exclusion of IgG from the core of the vehicle is not the touchstone of immunoisolation, because, in most cases, IgG alone is insufficient to produce cytolysis of the target cells or tissues. Thus, for immunoisolatory capsules, jacket nominal MWCO values up to 1000 kD are contemplated. For example, the MWCO is between 50-700 kD or between 50-500 kD or between 70-300 kD. See, e.g., WO 92/19195. In one embodiment, the MWCO is 500 kD.

The instant invention also relates to biocompatible, optionally immunoisolatory and/or immunoprotective, cryopreserved devices for the delivery of biologically active molecule(s) to the eye. Such devices contain a core containing a cryoprotectant and living cells that produce or secrete the biologically active molecule(s) and a biocompatible jacket surrounding the core, wherein the jacket has a MWCO that allows the diffusion of the biologically active molecule(s) into the eye and to the central nervous system, including the brain, ventricle, spinal cord.

The invention also provides biocompatible and implantable and optionally immunoisolatory and/or immunoprotective cryopreserved devices, containing a core having a cryoprotectant and cells that produces or secretes one or more biologically active molecule(s) and a semi-permeable membrane surrounding the cells, which permits the diffusion of the one or more biologically active molecules there through.

Those skilled in the art will recognize that any device configuration(s) can be cryopreserved in accordance with the instant invention. The device can be any configuration appropriate for maintaining biological activity and providing access for the delivery of the biologically active molecule. The particular device configuration(s) used will not impact the beneficial effects associated with cryopreservation.

By way of non-limiting example, suitable devices may include one, two, three, four, five, six, seven or all of the following additional characteristics:
a. the core contains about 0.5-1.0×10⁶ ARPE-19 cells;
b. the length of the device is about 1 mm-20 mm;
c. the internal diameter of the device is between 0.1 mm-2 0 mm;
d. the ends of the device are sealed using methyl methacrylate;
e. the semi-permeable membrane has a median pore size of about 100 nm;
f. the nominal MWCO of the semi-permeable membrane is between 50 and 500 kD;
g. the semi-permeable membrane is between 90-120 um thick;
h. the core contains an internal scaffold, wherein the scaffold comprises polyethylene terephthalate (PET) fibers that comprises between 40-85% of internal volume of the device;
i. any combination(s) thereof.

A variety of biocompatible capsules are suitable for delivery of molecules according to this invention. Useful biocompatible polymer capsules comprise (a) a core which contains a cell or cells, either suspended in a liquid medium or immobilized within a biocompatible matrix, and (b) a surrounding jacket comprising a membrane which does not contain isolated cells, which is biocompatible, and permits diffusion of the cell-produced biologically active molecule into the eye. Those skilled in the relevant art will be about to select the appropriate device configuration for a given indication or use without undue experimentation.

Many transformed cells or cell lines are advantageously isolated within a capsule having a liquid core, comprising, e.g., a nutrient medium, and optionally containing a source of additional factors to sustain cell viability and function. The core of the devices of the invention can function as a reservoir for growth factors (e.g., prolactin, or insulin-like growth factor 2), growth regulatory substances such as transforming growth factor β (TGF-β) or the retinoblastoma gene protein or nutrient-transport enhancers (e.g., perfluorocarbons, which can enhance the concentration of dissolved oxygen in the core). Certain of these substances are also appropriate for inclusion in liquid media.

Alternatively, the core may comprise a biocompatible matrix of a hydrogel or other biocompatible material (e.g., extracellular matrix components) which stabilizes the position of the cells. Any suitable matrix or spacer may be employed within the core, including precipitated chitosan, synthetic polymers and polymer blends, microcarriers and the like, depending upon the growth characteristics of the cells to be encapsulated.

Alternatively, the devices may have an internal scaffold. The scaffold may prevent cells from aggregating and improve cellular distribution within the device. (See PCT publication no. WO 96/02646, incorporated herein by reference). The scaffold defines the microenvironment for the encapsulated cells and keeps the cells well distributed within the core. The optimal internal scaffold for a particular device is highly dependent on the cell type to be used. In the absence of such a scaffold, adherent cells aggregate to form clusters.

For example, the internal scaffold may be a yarn or a mesh. The filaments used to form a yarn or mesh internal scaffold are formed of any suitable biocompatible, substantially non-degradable material. (See U.S. Pat. Nos. 6,303,136 and 6,627,422, which are herein incorporated by reference). Materials useful in forming yarns or woven meshes include any biocompatible polymers that are able to be formed into fibers such as, for example, acrylic, polyester, polyethylene, polypropylene, polyacrylonitrile, polyethylene terephthalate, nylon, polyamides, polyurethanes, polybutester, or natural fibers such as cotton, silk, chitin or carbon.

In some embodiments, the filaments, which can be organized in a non-random unidirectional orientation, are twisted in bundles to form yarns of varying thickness and void volume. Void volume is defined as the spaces existing between filaments. The void volume in the yarn should vary between 20-95%, for example, between 50-95%. In one embodiment, the internal scaffold is made from PET fibers that fill between 40-85% of the internal volume of the devices. Alternatively, the filaments or yarns can be woven into a mesh. In other embodiments, a tubular braid is constructed.

For implant sites that are not immunologically privileged, such as periocular sites, and other areas outside the anterior chamber (aqueous) and the posterior chamber (vitreous), the capsules can be immunoisolatory. Components of the biocompatible material may include a surrounding semipermeable membrane and the internal cell-supporting scaffolding. The transformed cells can be seeded onto the scaffolding, which is encapsulated by the permselective membrane, which is described above. Also, bonded fiber structures can be used for cell implantation. (See U.S. Pat. No. 5,512,600, incorporated by reference). Biodegradable polymers include, for example, those comprised of poly(lactic acid) PLA, poly(lactic-coglycolic acid) PLGA, and poly(glycolic acid) PGA and their equivalents. Foam scaffolds have been used to provide surfaces onto which transplanted cells may adhere (PCT International patent application Ser. No. 98/05304, incorporated by reference). Woven mesh tubes have been used as vascular grafts (PCT International patent application WO 99/52573, incorporated by reference). Additionally, the core can be composed of an immobilizing matrix formed from a hydrogel, which stabilizes the position of the cells. A hydrogel is a 3-dimensional network of cross-linked hydrophilic polymers in the form of a gel, substantially composed of water.

Various polymers and polymer blends can be used to manufacture the surrounding semipermeable membrane, including polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), as well as derivatives, copolymers and mixtures thereof. In some illustrative embodiments, the surrounding semipermeable membrane is a biocompatible semipermeable hollow fiber membrane. Such membranes, and methods of making them are disclosed by U.S. Pat. Nos. 5,284,761 and 5,158,881, incorporated by reference. The surrounding semipermeable membrane is formed from a polyether sulfone hollow fiber, such as those described by U.S. Pat. No. 4,976,859 or U.S. Pat. No. 4,968,733, incorporated by reference. An alternate surrounding semipermeable membrane material is polysulfone.

The capsule can be any configuration appropriate for maintaining biological activity and providing access for delivery of the product or function and/or including for example, cylindrical, rectangular, disk-shaped, patch-shaped, ovoid, stellate, or spherical. Moreover, the capsule can be coiled or wrapped into a mesh-like or nested structure. If the capsule is to be retrieved after it is implanted, configurations which tend to lead to migration of the capsules from the site of implantation, such as spherical capsules small enough to travel in the recipient host's blood vessels, are not preferred. Certain shapes, such as rectangles, patches, disks, cylinders, and flat sheets offer greater structural integrity and can be used where retrieval is desired.

The device may have a tether that aids in maintaining device placement during implant, and aids in retrieval. Such a tether may have any suitable shape that is adapted to secure the capsule in place. For example, the tether may be a loop, a disk, or a suture. In some embodiments, the tether is shaped like an eyelet, so that a suture may be used to secure the tether (and thus the device) to the sclera, or other suitable ocular structure. In another embodiment, the tether is continuous with the capsule at one end, and forms a pre-threaded suture needle at the other end. In one embodiment, the tether is an anchor loop that is adapted for anchoring the capsule to an ocular structure. The tether may be constructed of a shape memory metal and/or any other suitable medical grade material known in the art.

In a hollow fiber configuration, the fiber will have an inside diameter of less than 2000 microns, for example, less than 1200 microns. Also contemplated are devices having an outside diameter less than 300-600 microns. In one embodiment, the inner diameter is between 0.1 mm and 2.0 mm For implantation in the eye, in a hollow fiber configuration the capsule can be between 0.4 cm to 1.5 cm in length or between 0.4 to 1.0 cm in length. In one embodiment, the length of the device is between 1 mm and 20 mm Longer devices may be accommodated in the eye, however, a curved or arcuate shape may be required for secure and appropriate placement. The hollow fiber configuration can be used for intraocular placement.

For periocular placement, either a hollow fiber configuration (with dimensions substantially as above) or a flat sheet configuration is contemplated. The upper limit contemplated for a flat sheet is approximately 5 mm×5 mm—assuming a square shape. Other shapes with approximately the same surface area are also contemplated.

Microdevices manufactured for delivery of the anti-angiogenic antibody-scaffold, soluble VEGFR or soluble PDGFR or one or more biologically active molecule(s) may have a length of between 1 and 2 5 millimeters, with an inner diameter of between 300 and 500 microns and an outer diameter of between 450 and 700 microns. The internal volume of a micronized device will be less than 0.5 μl (i.e., 0.5 μl). For a complete discussion of micronized devices, see WO2007/078922, which is herein incorporated by reference.

The open membrane contemplated for use will have nominal molecular weight cutoff (MWCO) values up to 1000 kD. For example, the MWCO is between 50-700 kD or between 50-500 kD and ideally approximately 300 kD. In one embodiment, the MWCO is 500 kD. The nominal pore size of the membrane contemplated will have a nominal pore size of approximately 100 nm and based upon a Gaussian distribution of pores the largest absolute pores would be less than 150 nm. Alternatively, if a very open membrane is not utilized, a more “immunoisolatory” and/or “immunoprotective” membrane will be used.

In one embodiment, the median pore size is about 100 nm. The surrounding or peripheral region (jacket), which surrounds the core of the instant devices can be permselective, biocompatible, and/or immunoisolatory. It is produced in such a manner that it is free of isolated cells, and completely surrounds (i.e., isolates) the core, thereby preventing contact between any cells in the core and the recipient's body. Biocompatible semi-permeable hollow fiber membranes, and methods of making them are disclosed in U.S. Pat. Nos. 5,284,761 and 5,158,881 (See also, WO 95/05452), each of which incorporated herein by reference in its entirety. For example, the capsule jacket can be formed from a polyether sulfone hollow fiber, such as those described in U.S. Pat. Nos. 4,976,859 and 4,968,733, and 5,762,798, each incorporated herein by reference.

To be permselective, the jacket is formed in such a manner that it has a MWCO range appropriate both to the type and extent of immunological reaction anticipated to be encountered after the device is implanted and to the molecular size of the largest substance whose passage into and out of the device into the eye is desirable. The type and extent of immunological attacks which may be mounted by the recipient following implantation of the device depend in part upon the type(s) of moiety isolated within it and in part upon the identity of the recipient (i.e., how closely the recipient is genetically related to the source of the BAM). When the implanted tissue or cells are allogeneic to the recipient, immunological rejection may proceed largely through cell-mediated attack by the recipient's immune cells against the implanted cells. When the tissue or cells are xenogeneic to the recipient, molecular attack through assembly of the recipient's cytolytic complement attack complex may predominate, as well as the antibody interaction with complement.

The jacket allows passage of substances up to a predetermined size, but prevents the passage of larger substances. More specifically, the surrounding or peripheral region is produced in such a manner that it has pores or voids of a predetermined range of sizes, and, as a result, the device is permselective. The MWCO of the surrounding jacket must be sufficiently low to prevent access of the substances required to carry out immunological attacks to the core, yet sufficiently high to allow delivery biologically active molecule(s) to the recipient. When truncated biologically active molecule(s) are used, the MWCO of the biocompatible jacket of the devices of the instant invention is from about 1 kD to about 150 kD. However, if delivery of a non-truncated biologically active molecule(s) is desired, an open membrane with a MWCO greater than 200 kD should be used.

As used herein with respect to the jacket of the device, the term “biocompatible” refers collectively to both the device and its contents. Specifically, it refers to the capability of the implanted intact device and its contents to avoid the detrimental effects of the body's various protective systems and to remain functional for a significant period of time. As used herein, the term “protective systems” refers to the types of immunological attack which can be mounted by the immune system of an individual in whom the instant vehicle is implanted, and to other rejection mechanisms, such as the fibrotic response, foreign body response and other types of inflammatory response which can be induced by the presence of a foreign object in the individuals' body. In addition to the avoidance of protective responses from the immune system or foreign body fibrotic response, the term “biocompatible”, as used herein, also implies that no specific undesirable cytotoxic or systemic effects are caused by the vehicle and its contents such as those that would interfere with the desired functioning of the vehicle or its contents.

The external surface of the device can be selected or designed in such a manner that it is particularly suitable for implantation at a selected site. For example, the external surface can be smooth, stippled or rough, depending on whether attachment by cells of the surrounding tissue is desirable. The shape or configuration can also be selected or designed to be particularly appropriate for the implantation site chosen.

The choice of materials used to construct the device is determined by a number of factors as described in detail in Dionne WO 92/19195, herein incorporated by reference. Briefly, various polymers and polymer blends can be used to manufacture the capsule jacket. Polymeric membranes forming the device and the growth surfaces therein may include polyacrylates (including acrylic copolymers), polyvinylidenes, polyvinyl chloride copolymers, polyurethanes, polystyrenes, polyamides, polymethylmethacrylate, polyvinyldifluoride, polyolefins, cellulose acetates, cellulose nitrates, polysulfones, polyphosphazenes, polyacrylonitriles, poly(acrylonitrile/covinyl chloride), as well as derivatives, copolymers and mixtures thereof.

In some embodiments, the jacket of the present device is immunoisolatory and/or immunoprotective. That is, it protects cells in the core of the device from the immune system of the individual in whom the device is implanted. It does so (1) by preventing harmful substances of the individual's body from entering the core, (2) by minimizing contact between the individual and inflammatory, antigenic, or otherwise harmful materials which may be present in the core and (3) by providing a spatial and physical barrier sufficient to prevent immunological contact between the isolated moiety and detrimental portions of the individual's immune system.

In some embodiments, the external jacket may be either an ultrafiltration membrane or a microporous membrane. Those skilled in the art will recognize that ultrafiltration membranes are those having a pore size range of from about 1 to about 100 nanometers while a microporous membrane has a range of between about 1 to about 10 microns.

The thickness of this physical barrier can vary, but it will always be sufficiently thick to prevent direct contact between the cells and/or substances on either side of the barrier. The thickness of this region generally ranges between 5 and 200 microns. For example, thicknesses of 10 to 100 microns or of 20 to 50 or 20 to 75 microns can be used. In one embodiment, the semi-permeable membrane is between 90 and 120 μm thick. Types of immunological attack which can be prevented or minimized by the use of the instant device include attack by macrophages, neutrophils, cellular immune responses (e.g. natural killer cells and antibody-dependent T cell-mediated cytolysis (ADCC)), and humoral response (e.g. antibody-dependent complement mediated cytolysis).

The capsule jacket may be manufactured from various polymers and polymer blends including polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), as well as derivatives, copolymers and mixtures thereof. Capsules manufactured from such materials are described, e.g., in U.S. Pat. Nos. 5,284,761 and 5,158,881, incorporated herein by reference. Capsules formed from a polyether sulfone (PES) fiber, such as those described in U.S. Pat. Nos. 4,976,859 and 4,968,733, incorporated herein by reference, may also be used.

Those skilled in the art will recognize that capsule jackets with permselective, immunoisolatory membranes are preferable for sites that are not immunologically privileged.

In contrast, microporous membranes or permselective membranes may be suitable for immunologically privileged sites. For implantation into immunologically privileged sites, capsules made from the PES or PS membranes can be used.

The methods and devices of this invention are intended for use in a primate, for example, a human host, recipient, patient, subject or individual. A number of different ocular implantation sites are contemplated for the devices and methods of this invention. Suitable implantation sites include, but are not limited to, the aqueous and vitreous humors of the eye, the periocular space, the anterior chamber, and/or the Subtenon's capsule. Within the body, implantation sites may include subcutaneous, intraperitoneal, or within the CNS. In addition, implantation may be directed at localized delivery at or near lesions requiring the desired biologic therapy. Example of such disease sites may be inflamed joints, brain and CNS lesions, sites of benign or malignant tumors. Access by the device to the circulatory system can further extend the range of potential disease sites within the body to distally affected organs and tissues.

The type and extent of immunological response by the recipient to the implanted device will be influenced by the relationship of the recipient to the isolated cells within the core. For example, if core contains syngeneic cells, these will not cause a vigorous immunological reaction, unless the recipient suffers from an autoimmunity with respect to the particular cell or tissue type within the device. Syngeneic cells or tissue are rarely available. In many cases, allogeneic or xenogeneic cells or tissue (i.e., from donors of the same species as, or from a different species than, the prospective recipient) may be available. The use of immunoisolatory devices allows the implantation of allogeneic or xenogeneic cells or tissue, without a concomitant need to immunosuppress the recipient. Use of immunoisolatory capsules also allows the use of unmatched cells (allographs). Therefore, the instant device makes it possible to treat many more individuals than can be treated by conventional transplantation techniques.

Capsules with a lower MWCO may be used to further prevent interaction of molecules of the patient's immune system with the encapsulated cells.

Any of the devices used in accordance with the methods described herein must provide, in at least one dimension, sufficiently close proximity of any isolated cells in the core to the surrounding tissues (i.e., eye tissues) of the recipient in order to maintain the viability and function of the isolated cells.

The device of the present invention is of a sufficient size and durability for complete retrieval after implantation. In one example, the device has a core of a volume of approximately 2-20 μL (e.g., 1-3 μL). The internal geometry of micronized devices has a volume of approximately 0.05-0.1 γL. Other device configuration and/or geometries can also be employed.

According to the methods of this invention, other molecules (e.g., additional biologically active molecules (“BAMs”)) may be co-delivered. For example, trophic factor(s) may be delivered with anti-angiogenic factor(s).

Co-delivery can be accomplished in a number of ways. First, cells may be transfected with separate constructs containing the genes encoding the described molecules. Second, cells may be transfected with a single construct containing two or more genes as well as the necessary control elements. Third, two or more separately engineered cell lines can be either co-encapsulated or more than one device can be implanted at the site of interest.

For some indications, BAMs may be delivered to two different sites (e.g., in the eye) concurrently. For example, it may be desirable to deliver a neurotrophic factor to the vitreous to supply the neural retina (ganglion cells to the RPE) and to deliver an anti-angiogenic factor (such as one or more of the soluble receptors or anti-angiogenic antibodies and molecules) via the sub-Tenon's space to supply the choroidal vasculature.

This invention also contemplates use of different cell types during the course of the treatment regime. For example, a patient may be implanted with a capsule device containing a first cell type (e.g., BHK cells). If after time, the patient develops an immune response to that cell type, the capsule can be retrieved, or explanted, and a second capsule can be implanted containing a second cell type (e.g., CHO cells). In this manner, continuous provision of the therapeutic molecule is possible, even if the patient develops an immune response to one of the encapsulated cell types.

Along with the biologically active molecule(s) described herein, at least one additional BAM can also be delivered from the device to the eye. For example, the at least one additional BAM can be provided from a cellular or a noncellular source. When the at least one additional BAM is provided from a noncellular source, the additional BAM(s) may be encapsulated in, dispersed within, or attached to one or more components of the cell system including, but not limited to: (a) sealant; (b) scaffold; (c) jacket membrane; (d) tether anchor; and/or (e) core media. In such embodiment, co-delivery of the additional BAM(s) from a noncellular source may occur from the same device as the BAM from the cellular source.

Alternatively, two or more encapsulated cell systems can be used. For example, the least one additional biologically active molecule can be a nucleic acid, a nucleic acid fragment, a peptide, a polypeptide, a peptidomimetic, a carbohydrate, a lipid, an organic molecule, an inorganic molecule, a therapeutic agent, or any combinations thereof. Specifically, the therapeutic agents may be an anti-angiogenic drug, a steroidal and non-steroidal anti-inflammatory drug, an anti-mitotic drug, an anti-tumor drug, an anti-parasitic drug, an IOP reducer, a peptide drug, and/or any other biologically active molecule drugs approved for commercial use.

Suitable excipients include, but are not limited to, any non-degradable or biodegradable polymers, hydrogels, solubility enhancers, hydrophobic molecules, proteins, salts, or other complexing agents approved for formulations.

Non-cellular dosages can be varied by any suitable method known in the art such as varying the concentration of the therapeutic agent, and/or the number of devices per eye, and/or modifying the composition of the encapsulating excipient. Cellular dosage can be varied by changing (1) the number of cells per device, (2) the number of devices per eye, and/or (3) the level of BAM production per cell (e.g., by iterative transfection). Cellular production can be varied by changing, for example, the copy number of the gene for the additional BAM(s) in the transduced cell, or the efficiency of the promoter driving expression of the additional BAM(s). Suitable dosages from cellular sources may range from about 1 pg to about 10000 mg per day.

Devices may be formed by any suitable method known in the art. (See, e.g.,U.S. Pat. Nos. 6,361,771; 5,639,275; 5,653,975; 4,892,538; 5,156,844; 5,283,138; and 5,550,050, each of which is incorporated herein by reference).

Any suitable method of sealing the capsules know in the art may be used, including the employment of polymer adhesives and/or crimping, knotting and heat sealing. In addition, any suitable “dry” sealing method can also be used. In such methods, a substantially non-porous fitting is provided through which the cell-containing solution is introduced. Subsequent to filling, the capsule is sealed. Such methods are described in, e.g., U.S. Pat. Nos. 5,653,688; 5,713,887; 5,738,673; 6,653,687; 5,932,460; and 6,123,700, which are herein incorporated by reference. In one method, the ends of the device are sealed using methyl methacrylate.

Membranes used can also be tailored to control the diffusion of biologically active molecules, based on their molecular weight. (See Lysaght et al., 56 J. Cell Biochem. 196 (1996), Colton, 14 Trends Biotechnol. 158 (1996)). Using encapsulation techniques, cells can be transplanted into a host without immune rejection, either with or without use of immunosuppressive drugs. The capsule can be made from a biocompatible material that, after implantation in a host, does not elicit a detrimental host response sufficient to result in the rejection of the capsule or to render it inoperable, for example through degradation.

The number of devices and device size should be sufficient to produce a therapeutic effect upon implantation and is determined by the amount of biological activity required for the particular application. In the case of secretory cells releasing therapeutic substances, standard dosage considerations and criteria known to the art will be used to determine the amount of secretory substance required. Factors to be considered include the size and weight of the recipient; the productivity or functional level of the cells; and, where appropriate, the normal productivity or metabolic activity of the organ or tissue whose function is being replaced or augmented. It is also important to consider that a fraction of the cells may not survive the immunoisolation and implantation procedures. Moreover, whether the recipient has a preexisting condition which can interfere with the efficacy of the implant must also be considered. Devices of the instant invention can easily be manufactured which contain many thousands of cells. For example, current ophthalmic clinical devices contain between 200,000 and 750,000 cells, whereas micronized devices would contain between 10,000 and 100,000 cells. Other large scale devices may contain between 1,000,000 to 100,000,000 cells.

Encapsulated cell therapy is based on the concept of isolating cells from the recipient host's immune system by surrounding the cells with a semipermeable biocompatible material before implantation within the host. For example, the invention includes a device (e.g., a cryopreserved device) in which genetically engineered ARPE-19 cells are encapsulated in an immunoisolatory capsule, which, upon implantation into a recipient host, minimizes the deleterious effects of the host's immune system on the ARPE-19 cells in the core of the device. ARPE-19 cells are immunoisolated from the host by enclosing them within implantable polymeric capsules formed by a microporous membrane. This approach prevents the cell-to-cell contact between the host and implanted tissues, thereby eliminating antigen recognition through direct presentation.

Any of the biologically active molecule(s) secreted by the devices described herein (alone or in any combination) can be delivered intraocularly (e.g., in the anterior chamber and the vitreous cavity), periocularly (e.g., within or beneath Tenon's capsule), or both. The devices of the invention may also be used to provide controlled and sustained release of the biologically active molecules to treat various ophthalmic disorders, ophthalmic diseases, and/or other diseases which have ocular effects.

Treatment of many conditions according to the methods and uses described herein will required only one or at most less than 50 implanted devices per eye to supply an appropriate therapeutic dosage.

Intraocular (e.g., in the vitreous) or per ocular (e.g., in the sub-Tenon's space or region) allow for the delivery of a biologically active molecule(s). Therapeutic dosages may be between 0.1 pg and 10000 μg (e.g., between 0.1 pg and 5000 μg; between 0.1 pg and 2500 μg; between 0.1 pg and 1000 μg; between 0.1 pg and 500 μg; between 0.1 pg and 250 μg;

between 0.1 pg and 100 μg; between 0.1 pg and 50 μg; between 0.1 pg and 25 μg; between 0.1 pg and 10 μg; between 0.1 pg and 5 μg; between 0.1 pg and 100 ng; between 0.1 pg and 50 ng; between 0.1 pg and 25 ng; between 0.1 pg and 10 ng; or between 0.1 pg and 5 ng) per eye per patient per day is contemplated. In one non-limiting example, the therapeutic amount is at least 0.5-50 μg/ml steady state in the eye. Suitable therapeutic amounts may include, for example, 0.5 μg, 0.6 μg, 0.7 ug, 0.8 μg, 0.9 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 1500 μg, 2000 μg, 2500 μg, 3000 μg, 3500 μg, 4000 μg, 4500 μg, 5000 μg, 5500 μg, 6000 μg, 6500 μg, 7000 μg, 7500 μg, 8000 μg, 8500 μg, 9000 μg, 9500 μg, 10000 μg. Moreover, the cells lines and devices of the instant invention are able to express this therapeutic amount for a period of at least three months.

Ophthalmic disorders that may be treated by various embodiments of the present invention include, but are not limited to diabetic retinopathies, diabetic macular edema, proliferative retinopathies, retinal vascular diseases, vascular anomalies, age-related macular degeneration and other acquired disorders, endophthalmitis, infectious diseases, inflammatory but non-infectious diseases, AIDS-related disorders, ocular ischemia syndrome, pregnancy-related disorders, peripheral retinal degenerations, retinal degenerations, toxic retinopathies, retinal tumors, choroidal tumors, choroidal disorders, vitreous disorders, retinal detachment and proliferative vitreoretinopathy, non-penetrating trauma, penetrating trauma, post-cataract complications, and inflammatory optic neuropathies.

Those skilled in the art will recognize that age-related macular degeneration includes, but is not limited to, wet and dry age-related macular degeneration, exudative age-related macular degeneration, and myopic degeneration.

In some embodiments, the disorder to be treated is the wet form of age-related macular degeneration or diabetic retinopathy. The present invention may also be useful for the treatment of ocular neovascularization, a condition associated with many ocular diseases and disorders. For example, retinal ischemia-associated ocular neovascularization is a major cause of blindness in diabetes and many other diseases.

The cell lines and cryopreserved devices of the present invention may also be used to treat ocular symptoms resulting from diseases or conditions that have both ocular and non-ocular symptoms. Some examples include cytomegalovirus retinitis in AIDS as well as other conditions and vitreous disorders; hypertensive changes in the retina as a result of pregnancy; and ocular effects of various infectious diseases such as tuberculosis, syphilis, Lyme disease, parasitic disease, toxocara canis, ophthalmonyiasis, cyst cercosis and fungal infections.

The devices and cell lines may also be used to treat conditions relating to other intraocular neovascularization-based diseases. For example, such neovascularization can occur in diseases such as diabetic retinopathy, central retinal vein occlusion and, possibly, age-related macular degeneration. Corneal neovascularization is a major problem because it interferes with vision and predisposes patients to corneal graft failure. A majority of severe visual loss is associated with disorders that result in ocular neovascularization.

The invention also relates to methods for the delivery of cytokines, neurotrophic factors, soluble receptors, anti-angiogenic antibodies and molecules or biologically active molecule(s) in order to treat cell proliferative disorders, such as, for example, hematologic disorders, atherosclerosis, inflammation, increased vascular permeability, and malignancy within the ocular environment or outside at desired targeted locations within the body.

The use of the devices and techniques described herein provide several advantages over other delivery routes: biologically active molecule(s) can be delivered to the eye directly, which reduces or minimizes unwanted peripheral side effects and very small doses of the biologically active molecule(s) (i.e., nanogram or low microgram quantities rather than milligrams) can be delivered compared with topical applications, thereby also potentially lessening side effects. Moreover, since viable cells continuously produce newly synthesized biologically active molecule(s), these techniques should be superior to injection delivery of the biologically active molecule(s), where the dose fluctuates greatly between injections and the biologically active molecule(s) is continuously degraded but not continuously replenished.

Living cells and cell lines genetically engineered to secrete the biologically active molecule(s) can be encapsulated in the device of the invention and surgically inserted (under retrobulbar anesthesia) into any appropriate anatomical structure of the eye. For example, the devices can be surgically inserted into the vitreous of the eye, where they are may be tethered to the sclera to aid in removal. Devices can remain in the vitreous as long as necessary to achieve the desired prophylaxis or therapy. For example, the desired therapy may include promotion of neuron or photoreceptor survival or repair, or inhibition and/or reversal of retinal or choroidal neovascularization, as well as inhibition of uveal, retinal and optic nerve inflammation. With vitreal placement, the biologically active molecule(s), may be delivered to the retina or the retinal pigment epithelium (RPE).

In other embodiments, cell-loaded devices are implanted periocularly, within or beneath the space known as Tenon's capsule, which is less invasive than implantation into the vitreous. Therefore, complications such as vitreal hemorrhage and/or retinal detachment are potentially eliminated. This route of administration also permits delivery of the biologically active molecule(s) described herein to the RPE or the retina. Periocular implantation can be used for treating choroidal neovascularization and inflammation of the optic nerve and uveal tract. In general, delivery from periocular implantation sites will permit circulation of the biologically active molecule(s) to the choroidal vasculature, retinal vasculature, and the optic nerve.

Delivery of biologically active molecule(s), such as the anti-angiogenic antibody-scaffolds or soluble VEGF receptors or PDGF receptors directly to the choroidal vasculature (periocularly) or to the vitreous (intraocularly) using the devices and methods described herein may reduce or alleviate the problems associated with prior art treatment methods and devices and may permit the treatment of poorly defined or occult choroidal neovascularization as well as provide a way of reducing or preventing recurrent choroidal neovascularization via adjunctive or maintenance therapy.

Following thawing, implantation of the cryopreserved biocompatible devices of the invention is performed under sterile conditions. The device can be implanted using a syringe or any other method known to those skilled in the art. Generally, the device is implanted at a site in the recipient's body which will allow appropriate delivery of the secreted product or function to the recipient and of nutrients to the implanted cells or tissue, and will also allow access to the device for retrieval and/or replacement. A number of different implantation sites are contemplated. These include, e.g., the aqueous humor, the vitreous humor, the sub-Tenon's capsule, the periocular space, and the anterior chamber. For implant sites that are not immunologically privileged, such as periocular sites, and other areas outside the anterior chamber (aqueous) and the posterior chamber (vitreous), the capsules are immunoisolatory.

It is preferable to verify that the cells immobilized within the device function properly both before and after implantation. Any assays or diagnostic tests well known in the art can be used for these purposes. For example, an ELISA (enzyme-linked immunosorbent assay), chromatographic or enzymatic assay, or bioassay specific for the secreted biologically active molecule(s) can be used. If desired, secretory function of an implant can be monitored over time by collecting appropriate samples (e.g., serum) from the recipient and assaying them.

The invention will be further described in the following examples, which do not limit the scope of the invention described in the claims.

### EXAMPLES

### Example 1

### Device Characterization and Implantation Results

### Cell Line Stability Studies

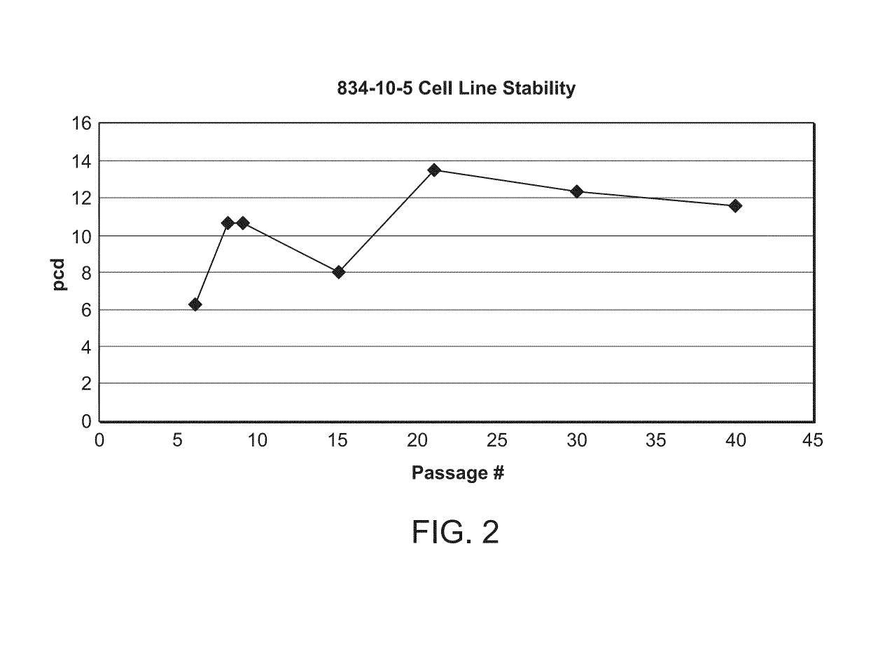

One criterion for manufacturability of recombinant cell lines is the limit of productivity by clonal expansion. It was calculated that growth and productivity analysis of 40 generations of clonal cells would confirm output stability, and supply sufficient information for the creation of a master cell bank (by passage ˜17) and a working cell bank (by passage ˜23). One working cell bank is calculated to be sufficient for the manufacture of at least 100,000,000 devices. Serial passage of p834-10-5 cell line revealed stability out to 40 generations in tissue culture with an average output of 10.4 pcd (picogram/cell/day) over the set of time points assayed. (See FIG. 2).

### Device Output Timecourse Studies

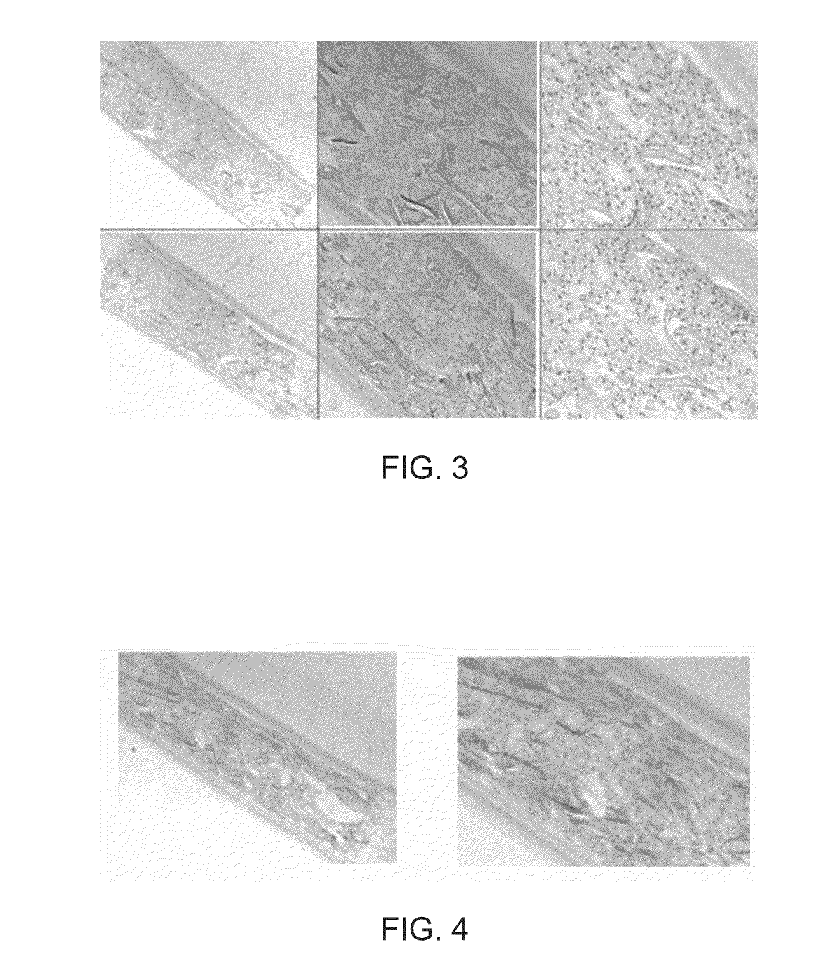

p834-10-5 cell lines were expanded from a research cell bank aliquot and expanded prior to device filling. Cells were encapsulated by injection into 6 mm ECT devices with walls constructed with polysulfone semipermeable membranes and filled with polyethylene terephthalate (PET) yarn for cellular attachment. Devices were individually placed into primary packaging of sealed containers with nutrient media and incubated at 37 C for 10 weeks. During the time course of incubation hold, recombinant protein output was periodically surveyed from ECT devices by removal from packaging and assay for p834-10-5 protein secretion by ELISA. Results show an initial device output of 480 ng/device/day of p834-10-5 protein, gradually tapering off to a baseline output of ˜60 ng/device/day after 6 weeks. In FIG. 3, histological sections of two devices reveal robust 834-10-5 cell growth internally, demonstrating high viability through one month device culture

Devices containing ARPE-19 cells genetically engineered to secrete the p834 VEGFR constructs showed excellent safety profiles at 1 and 3 months post implant. Moreover, these devices (“the NT-503 devices”) are stable in vivo at 1 and 3 months post implant.

Table 1 shows the PK data for these NT-503 devices:

TABLE 1
NT-503 PK

| Cell Line | Device Output (ng/day) | Vitreous Levels (ng/ml) |
|---|---|---|
| p834 (4-week Held) | | |
| 1-month | 439 ± 127 | 803 ± 107 |
| 3-month | 300 ± 54 | 350 ± 111 |

Table 2 shows the results of the NT-503 device shelf stability:

TABLE 2
NT-503 Shelf-Life: in vivo Stability Demonstrated
4-Week Shelf-Life

| | In vitro | | Explant | Vitreous |
|---|---|---|---|---|
| Cell | Held | Pre-implant | 1 month in vivo | 1 month in vivo |
| Line | (Weeks) | (ng/day) | (ng/day) | (ng/ml) |
| p834 | 1 Week | 478 | 345 | 644 |
| | 4 Week | 74 | 501 | 518 |

Both in vitro device output and in vivo performance (as measured for vitreous levels) were maintained stable for the NT-503 devices. Moreover, an evaluation of in vitro hold periods and corresponding in vivo performance of implanted NT-503 devices have demonstrated a shelf life stability of up to 4 weeks duration.

Finally, if possible, continued efforts will be made to extend the shelf-life of the NT-503 devices beyond 4 weeks (i.e., by cryopreserving the devices in accordance with the instant invention).

### Example 2

### Animal Studies

At four weeks after packaging, devices were implanted into non-immunosuppressed New Zealand White rabbit eyes. To determine p834-10-5 output after one month and three month after implantation, animals were enucleated and concentrations of p834-10-5 were quantified from extracted vitreous and compared with explanted device productivity. At one month after implantation, explanted devices produced p834-10-5 protein at greater than 100 ng/ml/day with steady-state vitreous concentrations at greater than 250 ng/ml. At three months after implantation, explanted devices continued production at over 200 ng/ml while vitreous concentration were detected at over 700 ng/ml. (See Table 3). After one year, rabbits vitreous samples contained 350 ng/ml p834-10-5 protein, demonstrating continued production of recombinant receptor over the course of 12 months.

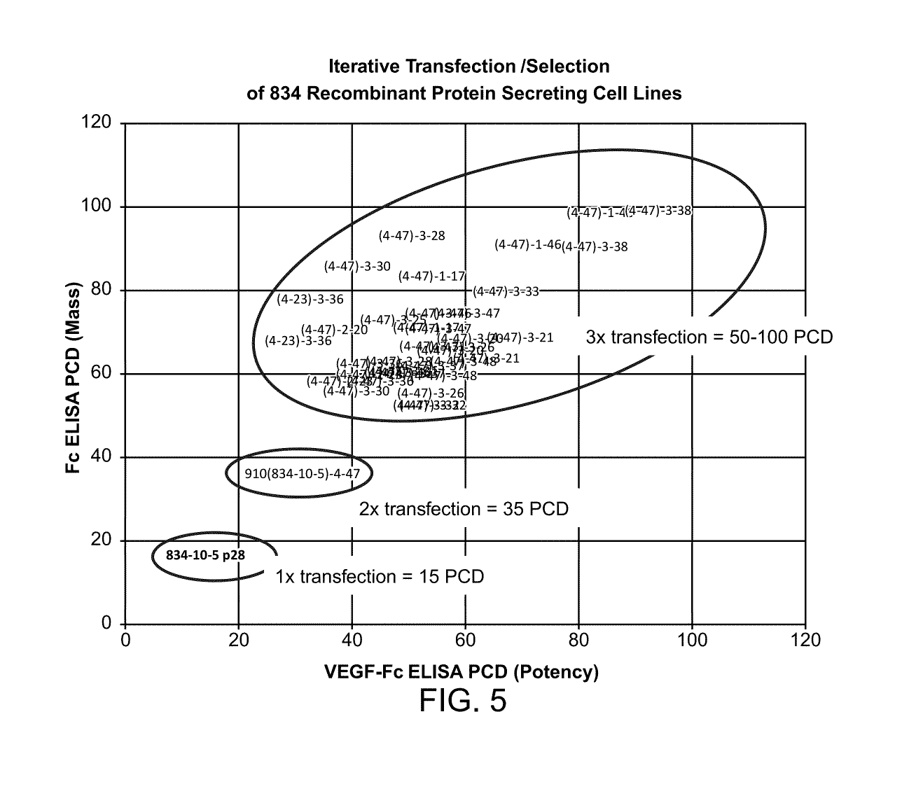

As shown in FIG. 4, histology of explanted devices after three months implantation revealed robust cell growth, analogous to the cellular morphology observed in sample from container-held devices shown in FIG. 3. No clinically significant adverse events were observed within the eye of the treated rabbits during the study, as periodically examined by a veterinary ophthalmologist.

TABLE 3
In vivo production of p834

| Sample | | |
|---|---|---|
| Identifier | | |
| | 1 Month | 1 Month |
| | Device Output | Vitreous Levels |
| | (ng/day) | (ng/ml) |
| Eye #1 | 250 | 760 |
| Eye #2 | 500 | 700 |
| Eye #3 | 482 | 800 |
| Eye #4 | 525 | 950 |
| | 3 Month | 3 Month |
| | Device Output | Vitreous Levels |
| | (ng/day) | (ng/ml) |
| Eye #5 | 350 | 200 |
| Eye #6 | 270 | 400 |
| Eye #7 | 340 | 340 |
| Eye #8 | 240 | 460 |

### Example 3

### Iterative Gene Dosing Increases Recombinant Protein Production

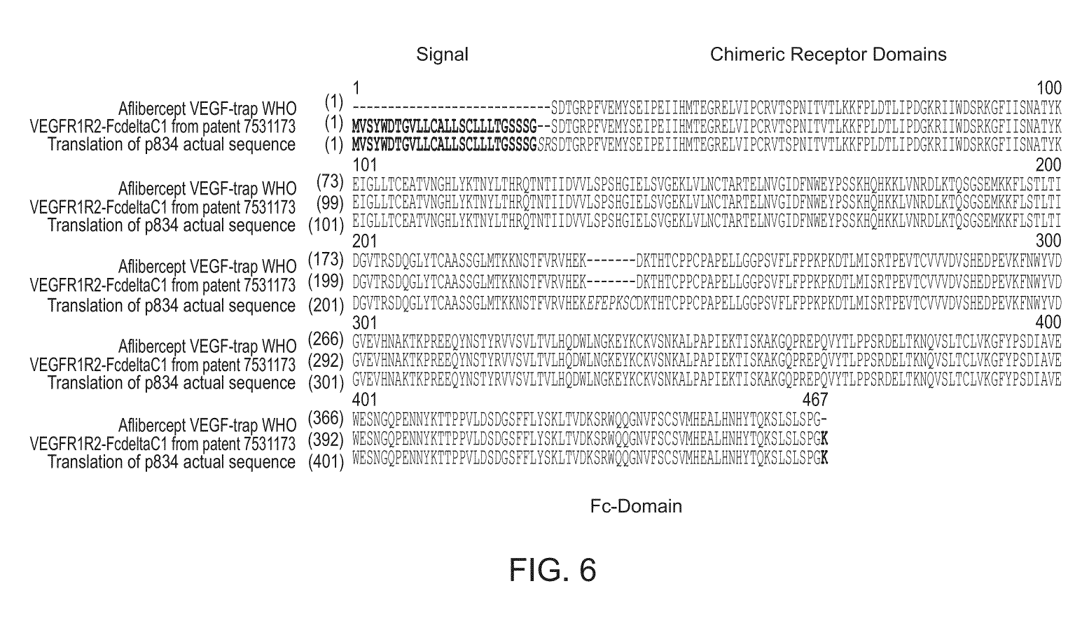

An iterative transfection was used to increase gene dosage, in particular of p834 cDNA. Three expression plasmids having identical 834 cDNA were produced: p834 pCpG vitro free (blasticidin resistant), p910 pCpG vitro free (neomycin resistant) and p969 pCpG vitro free (hygromycin resistant). p910 was transfected into blasticidin resistant p834-10-5 cell lines and resultant double integrant clones were recovered by application of neomycin selection, Subclones were isolated that exceeded PCD output levels of p834-10-5. As shown in FIG. 5, initial one time (“1×”) transfection yielded the aforementioned p834-10-5 cell line with naked cell output levels (Fc ELISA) at 15-20 PCD. Transfection and selection of p910 clones from parental 834-10-5 clones yielded 910 (834-10-5)-4-47 clones with output levels 35-40 PCD. Iterative transfection and selection of p969 into the 910 (834-10-5)-4-47 subclone yielded numerous hygromycin resistant p969 derived clones, with initial isolates secreting levels of recombinant protein ranging from 50 PCD to >100 PCD. Maintenance of expression from all three genetic integration events was confirmed by culture of 969 clonal lines in each of blasticidin, hygromycin, and neomycin culture medias. Triple transfection clones were present that demonstrated minimal loss in potency as determined by ELISA assays, based on direct binding of recombinant protein to plate bound VEGF followed by detection using anti-human Fc. Surprisingly, up to 8 fold higher values of recombinant protein was detected than simple arithmetic addition of gene dosage based on 3× transfection, suggesting that an unexpected, synergistic biological selection is involved with increasing gene dosage by serial transfection (e.g., using an iterative transfection process).

### Example 4

### Preclinical Studies of Dose Escalation by Iteratively Transfected Cell Lines

Following the method in Example 2, the double transfectant cell line 910(834-10-5)-4-47, and triple transfectant cell line 969(910(834-10-5)-4-47)-33, was used to generate ECT devices, and subsequently implanted into rabbits. After one month of implantation, rabbits were enucleated and vitreous were extracted to quantitate levels of 834 protein. Simultaneously, devices were surgically removed, and the explanted devices were further cultured in cell growth media to ascertain the device productivity of recombinant protein. As shown in Table 4, output from 910 and 969-devices resulted in the steady state vitreous levels of 834 protein at levels nearly 5 and 10-fold greater, respectively, than those observed with 834 single transfected protein (Table 4). Consistent with the cell line PCD output data, a higher steady state concentration of 834 protein was observed in vivo than expected by simple additive effect of serial transfected gene dose, (Table 5 vs. Table 3) again suggesting an unexpected, synergistic biological selection of synergistic secretion enhancement due to the iterative transfection methodology.

TABLE 4
in vivo production of p834 protein by 910(834-10-5)-4-47 devices

| Sample | 1 Month Device | 1 Month Vitreous |
|---|---|---|
| Identifier | Output (ng/day) | (ng/ml) |
| Eye# 9 | 1432 | 3641 |
| Fye# 10 | 2135 | 5572 |
| Eye# 11 | 2433 | 2710 |
| Eye# 12 | 1844 | 3603 |

TABLE 5
in vivo production of p834 protein by 969[910(834-10-5)-4-47]

| Sample | 1 Month Device | 1 Month Vitreous |
|---|---|---|
| Identifier | Output (ng/day) | Levels (ng/ml) |
| Eye #13 | 2511 | 9390 |
| Eye #14 | 3819 | 16031 |
| Eye #15 | 2055 | 7115 |
| Eye #16 | 2691 | 5680 |
| Eye #17 | 2145 | 10968 |
| Eye #18 | 2464 | 10840 |

### Example 5

### Cryopreservation of Encapsulated Cells

910(834-10-5)-4-47 cells were propagated in DMEM+10% FCS with seeding at 3×10⁶ cells per T-175 flasks. After 3 days of growth within a 5% CO2 and 37° C., humidity controlled incubator, the cells were trypsinized and resuspended to 100,000 cells/μl in Hyclone SFM4 MegaVir media, supplemented with 10 mM Glutamax and 10% glycerol as the cryoprotectant agent.

Cells were encapsulated by loading 8 mm ECT devices with 1×10⁶ cells followed by complete capsule closure. A total of 18 ECT devices were produced. ECT devices were then placed in 2 ml cryogenic vials containing 1 ml freezing media also containing the cryoprotectant agent. The cryogenic storage vials containing ECT devices were then frozen utilizing controlled rate freeze containers (Mr. Frosty or Cool Cell) rated at 1° C/minute, to a temperature of −80° C. After two days, the cryogenic vials were removed from -80° C. and immediately placed into liquid nitrogen storage under vapor phase.

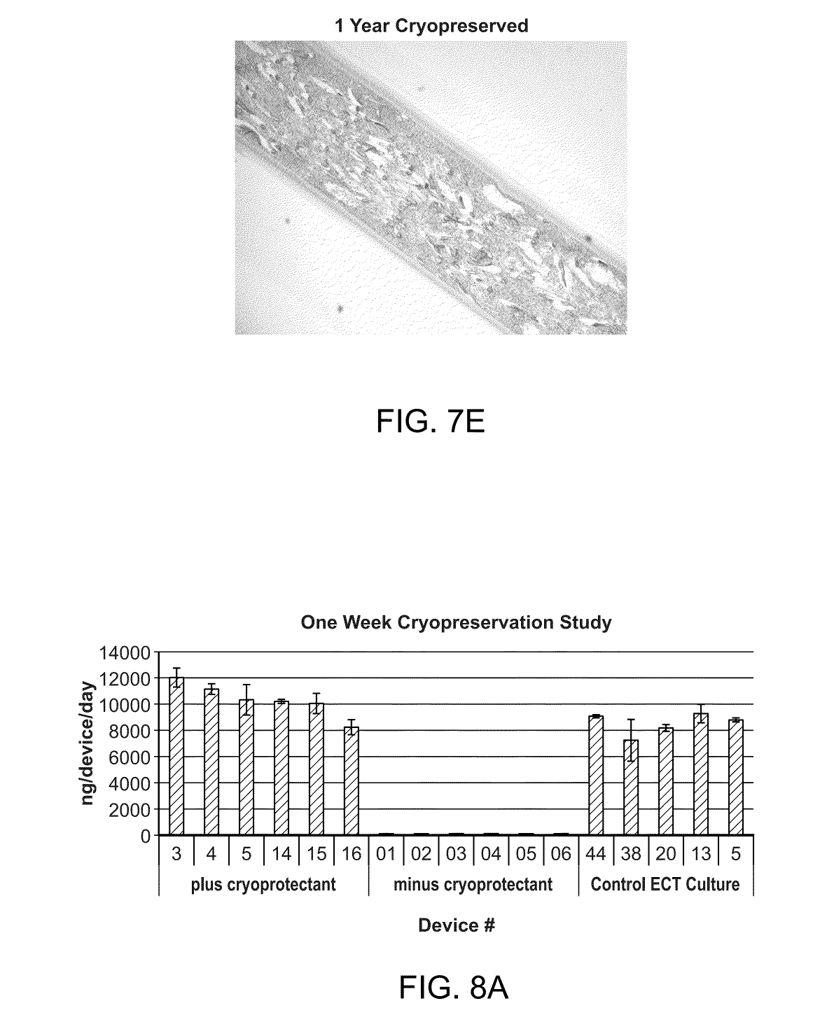

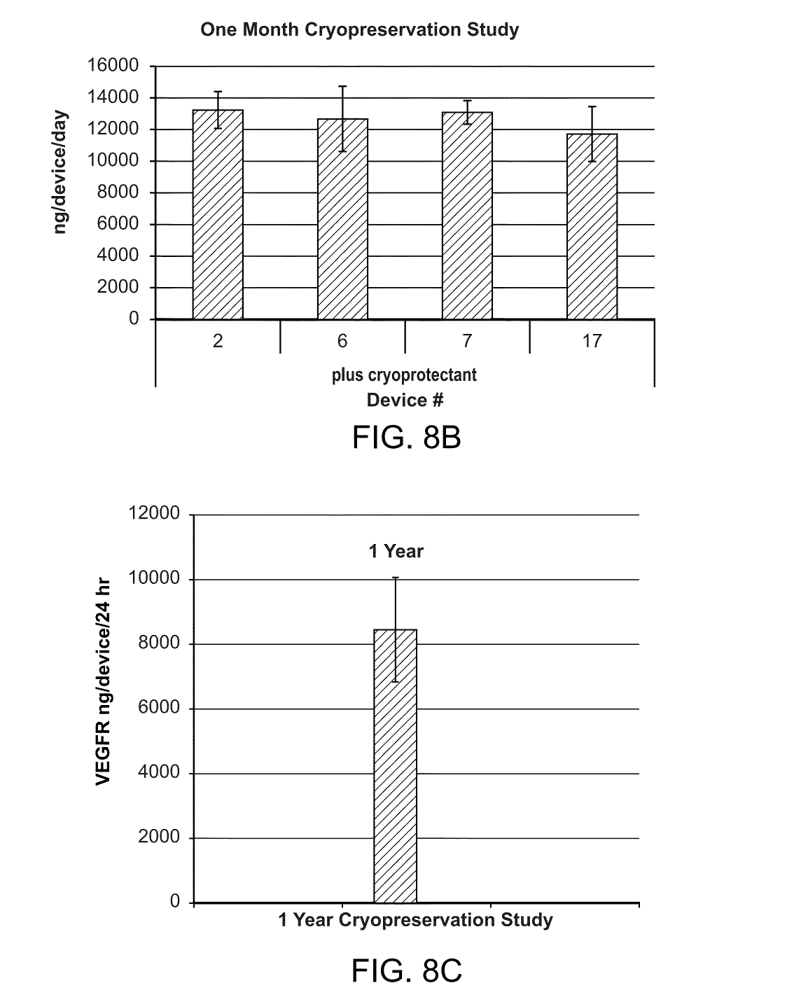

Cryogenic preserved implants were assessed at one week and one month, and one year post cryopreservation. At each time point, cryogenic vials were removed from vapor liquid nitrogen storage and ECT devices were placed into 37 ml of Hyclone SFM4 MegaVir media and held under standard tissue culture conditions. After 6 days, the ECT devices were assayed for cell confluence using CCK-8 colorimetric assay and VEGFR output was measured by Fc ELISA. ECT devices were subsequently fixed and histological sections stained for inspection of cell growth quality. Freezing of cells in the absence of cryoprotectant leads to cellular death (FIG. 7B), and the absence of VEGFR secretion from devices (FIG. 8A). Cryopreserved cells exhibit robust growth (FIGS. 7C, 7D, 7E) identical to normal culture ECT device (FIG. 7A), and high expression of VEGFR at each of the one week, one month, and one year post preservation time-points (FIGS. 8A, 8B, 8C). Cell viability, distribution and VEGFR secretion of the cryogenic preserved implants are at expected levels compared to the implants stored under conventional environmental controlled conditions.

### Equivalents

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but by the claims appended hereto.

1. A method of cryopreserving an implantable cell culture device, the device comprising:
a) a core comprising
(i) a cell line comprising an ARPE-19 cell genetically engineered to produce a therapeutically effective amount of one or more cytokines, neurotrophic factors, soluble receptors, anti-angiogenic antibodies and molecules, or biologically active molecules that are introduced into the ARPE-19 cell by an iterative transfection process, wherein the iterative transfection comprises one transfection, two transfections, or three transfections,
(ii) a cell line comprising an ARPE-19 cell genetically engineered to produce a therapeutically effective amount of one or more cytokines, neurotrophic factors, soluble receptors, anti-angiogenic antibodies and molecules, or biologically active molecules that at least 10,000 ng/day/10⁶ cells; or
(iii) ARPE-19 cells genetically engineered to secrete a therapeutically effective amount one or more biologically active molecules, and
b) a semi-permeable membrane surrounding the cell line in (i), the cell line in (ii), or the ARPE-19 cells in (iii), wherein the membrane permits the diffusion of the cytokines, neurotrophic factors, soluble receptors, anti-angiogenic antibodies and molecules, or biologically active molecules there through,
the method comprising the steps of:
adding a cryoprotectant agent to the core of the device,
placing the device in a cryogenic storage vial,
freezing the devices under controlled rate freezing, and
storing the device in dry ice (−70° C.), in a freezer (−80° C.), in vapor phase liquid nitrogen (−190° C.), or any combination thereof.

2. The method of claim 1, wherein the cryoprotectant agent is 10% glycerol.

3. The method of claim 21, wherein the controlled rate freezing occurs at −80° C.

4. The method of claim 31, wherein the method further comprises the step of transporting the devices under vapor phase liquid nitrogen (−190° C.) conditions, under dry ice (−70° C.) conditions, or a combination thereof.

5. The method of claim 1, wherein the cell line in (i) produces between 10,000 and 30,000 ng/day/10⁶cells of the one or more cytokines, neurotrophic factors, soluble receptors, anti-angiogenic antibodies and molecules, or biologically active molecules, when the iterative transfection is one transfection.

6. The method of claim 5, wherein the cell line in (i) produces about 15,000 ng/day/10⁶cells of the one or more cytokines, neurotrophic factors, soluble receptors, anti-angiogenic antibodies and molecules, or biologically active molecules.

7. The method of claim 1, wherein the cell line in (i) produces between 30,000 and 50,000 ng/day/10⁶cells of the one or more cytokines, neurotrophic factors, soluble receptors, anti-angiogenic antibodies and molecules, or biologically active molecules, when the iterative transfection is two transfections.

8. The method of claim 7, wherein the cell line in (i) produces about 35,000 ng/day/10⁶cells of the one or more cytokines, neurotrophic factors, soluble receptors, anti-angiogenic antibodies and molecules, or biologically active molecules.

9. The method of claim 1, wherein the cell line in (i) produces between 50,000 and 75,000 ng/day/10⁶cells of the one or more cytokines, neurotrophic factors, soluble receptors, anti-angiogenic antibodies and molecules, or biologically active molecules, when the iterative transfection is three transfections.

10. The method of claim 9, wherein the cell line in (i) produces about 70,000 ng/day/10⁶cells of the one or more cytokines, neurotrophic factors, soluble receptors, anti-angiogenic antibodies and molecules, or biologically active molecules.

11. The method of claim 1, wherein the one or more biologically active molecules is selected from the group consisting of neurotrophins, interleukins, cytokines, growth factors, anti-apoptotic factors, angiogenic factors, anti-angiogenic factors, antibodies and antibody fragments, antigens, neurotransmitters, hormones, enzymes, lymphokines, anti-inflammatory factors, therapeutic proteins, gene transfer vectors, brain derived neurotrophic factor (BDNF), NT-4, ciliary neurotrophic factor (CNTF), Axokine, basic fibroblast growth factor (bFGF), insulin-like growth factor I (IGF I), insulin-like growth factor II (IGF II), acid fibroblast growth factor (aFGF), epidermal growth factor (EGF), transforming growth factor a (TGF α), transforming growth factor β (TGF β), nerve growth factor (NGF), platelet derived growth factor (PDGF), glia-derived neurotrophic factor (GDNF), Midkine, phorbol 12-myristate 13-acetate, tryophotin, activin, thyrotropin releasing hormone, interleukins, bone morphogenic protein, macrophage inflammatory proteins, heparin sulfate, amphiregulin, retinoic acid, tumor necrosis factor α, fibroblast growth factor receptor, epidermal growth factor receptor (EGFR). PEDF, LEDGF, NTN, Neublastin, VEGF inhibitors, other agents expected to have therapeutically useful effects on potential target tissues, and any combination(s) thereof.

12-13. (canceled)

14. The method of claim 1 wherein the core further comprises a matrix disposed within the semi-permeable membrane.

15. The method of claim 14, wherein the matrix comprises a plurality of monofilaments, wherein said monofilaments are
a. twisted into a yarn or woven into a mesh, or
b. twisted into a yarn that is in non-woven stands, and wherein the cells are distributed thereon.

16. The method of claim 15, wherein the monofilaments comprise a biocompatible material selected from the group consisting of acrylic, polyester, polyethylene, polypropylene polyacetonitrile, polyethylene terephthalate, nylon, polyamides, polyurethanes, polybutester, silk, cotton, chitin, carbon, and biocompatible metals.

17. (canceled)

18. The method of claim 1, wherein the device further comprises a tether anchor.

19. The method of claim 18, wherein the tether anchor comprises an anchor loop.

20. The method of claim 19, wherein the anchor loop is adapted for anchoring the device to an ocular structure.

21-22. (canceled)

23. The method of claim 1, wherein the semi-permeable membrane comprises a permselective, immunoprotective membrane.

24. The method of claim 1, wherein the semi-permeable membrane comprises an ultrafiltration membrane, a microfiltration membrane, or a non porous membrane material.

25-26. (canceled)

27. The method of claim 24, wherein the non-porous membrane material is a hydrogel or a polyurethane.

28-29. (canceled)

30. The method of claim 1, wherein the device is configured as a hollow fiber or a flat sheet.

31-33. (canceled)

34. The method of claim 1, wherein at least one additional biologically active molecule is co-delivered from the device.

35. The method of claim 34, wherein the at least one additional biologically active molecule is from a non-cellular source or from a cellular source.

36. (canceled)

37. The method of claim 35, wherein the at least one additional biologically active molecule is produced by one or more genetically engineered ARPE-19 cells in the core.

38. The method of claim 1, wherein the device further comprises one or more additional characteristics selected from the group consisting of:
a. the core comprises between 0.5-1.0×10⁶ ARPE-19 cells;
b. length of the device is between 1 mm-20 mm;
c. the internal diameter of the device is between 0.1 mm-2.0 mm;
d. the ends of the device are sealed using methyl methacrylate;
e. the semi-permeable membrane has a median pore size of about 100 nm;
f. the nominal molecular weight cut off (MWCO) of the semi-permeable membrane is between 50 and 500 KD;
g. the semi-permeable membrane is between 90-120 μm thick;
h. the core comprises an internal scaffold, wherein the scaffold comprises polyethylene terephthalate (PET) fibers that comprise between 40-85% of the internal volume of the device; and
i. combinations thereof.

39. The method of claim 38, wherein the device comprises 2, 3, 4, 5, 6, 7, or all of the additional characteristics.

40-60. (canceled)

61. The method of claim 1, wherein the cryopreserved device is thawed prior to implantation.

62. The method of claim 61, wherein following thawing, the device is implanted into the eye of a patient.

63. The method of claim 62, wherein the device is implanted in the vitreous, the aqueous humor, the Subtenon's space, the periocular space, the posterior chamber, or the anterior chamber of the eye.
